SEQUENCE LISTING (53 sequences)

SEQ ID NO: 1 - protein, 9 aa, Homo Sapiens
CRRGQIKYS

SEQ ID NO: 2 - protein, 15 aa, Homo Sapiens
GPTNCRLRMHLGLVI

SEQ ID NO: 3 - protein, 12 aa, Homo Sapiens
RTWEEGKVLIFD

SEQ ID NO: 4 - protein, 12 aa, Homo Sapiens
WQDASSFRLIFI

SEQ ID NO: 5 - protein, 22 aa, Homo Sapiens
LSGTSFFTWFMVIALLGVWTSV

SEQ ID NO: 6 - protein, 28 aa, Homo Sapiens
VYEEVLSVTPNDGFAKVHYGFILKAQNK

SEQ ID NO: 7 - protein, 16 aa, Homo Sapiens
FASVWQRSLYNVNGLK

SEQ ID NO: 8 - protein, 24 aa, Homo Sapiens
GLSGTSFFTWFMVIALLGVWTSVA

SEQ ID NO: 9 - protein, 26 aa, Homo Sapiens
GDGDFDVDDAKVLLGLKERSTSEPAV

SEQ ID NO: 10 - protein, 28 aa, Homo Sapiens
IEEAVNAFKELVRKYPQSPRARYGKAQC

SEQ ID NO: 11 - protein, 23 aa, Homo Sapiens
DVPADLLKLSLKRRSDRQQFLGH

SEQ ID NO: 12 - protein, 23 aa, Homo Sapiens
SLERNWKLIRDEGLAVMDKAKGL

SEQ ID NO: 13 - protein, 22 aa, Homo Sapiens
GFAKVHYGFILKAQNKIAESIP

SEQ ID NO: 14 - protein, 22 aa, Homo Sapiens
HTGPTNCRLRMHLGLVIPKEGC

SEQ ID NO: 15 - protein, 23 aa, Homo Sapiens
DSFEHEVWQDASSFRLIFIVDVW

SEQ ID NO: 16 - protein, 23 aa, Homo Sapiens
MRGSLLTLQRLVQLFPNDTSLKN

SEQ ID NO: 17 - protein, 21 aa, Homo Sapiens
PQQEDDEFLMATDVDDRFETL

SEQ ID NO: 18 - protein, 21 aa, Homo Sapiens
DGRFYFHLGDAMQRVGNKEAY

SEQ ID NO: 19 - protein, 51 aa, Homo Sapiens
GLSGTSFFTWFMVIALLGVWTSVAGGSGGGFAKVHYGFILKAQNKIAESIP

SEQ ID NO: 20 - protein, 54 aa, Homo Sapiens
KVYEEVLSVEEVLSVTPNTPNDGFAKVGFAKVHYGFKVHYGFILKKIAESIPYL

SEQ ID NO: 21 - protein, 36 aa, Homo Sapiens
GTDDGRFYFRVGNKEAYKASVWQRSLYSLYNVNGLK

SEQ ID NO: 22 - protein, 19 aa, Homo Sapiens
SFFTWFMVIALLGVWTSVA

SEQ ID NO: 23 - protein, 18 aa, Homo Sapiens
PADLLKLSLKRRSDRQQF

SEQ ID NO: 24 - protein, 23 aa, Homo Sapiens
GFAKVHYGFILKAQNKIAESIPY

SEQ ID NO: 25 - protein, 21 aa, Homo Sapiens
TGPTNCRLRMHLGLVIPKEGC

SEQ ID NO: 26 - protein, 24 aa, Homo Sapiens
FEHEVWQDASSFRLIFVDVWHPEL

SEQ ID NO: 27 - protein, 26 aa, Homo Sapiens
EHVEGEDLQQEDGPTGEPQQEDDEFL

SEQ ID NO: 28 - protein, 18 aa, Homo Sapiens
PYLKEGIESGDPGTDDGR

SEQ ID NO: 29 - protein, 17 aa, Homo Sapiens
GLKAQPWWTPKETGYTE

SEQ ID NO: 30 - protein, 18 aa, Homo Sapiens
LKAQNKIAESIPYLKEGI

SEQ ID NO: 31 - protein, 31 aa, Homo Sapiens
HLGDAMQRVGNKEAYKWYELGHKRGHFASVW

SEQ ID NO: 32 - protein, 19 aa, Homo Sapiens
VDVWHPELTPQQRRSLPAI

SEQ ID NO: 33 - protein, 30 aa, Homo Sapiens
KNAKSSGNSSSSGSGSGSTSAGSSSPGARR

SEQ ID NO: 34 - protein, 32 aa, Homo Sapiens
IYDADGDGDFDVDDAKVLLGLKERSTSEPAVP

SEQ ID NO: 35 - protein, 18 aa, Homo Sapiens
EEMMSEQENPDSSEPVVE

SEQ ID NO: 36 - protein, 30 aa, Homo Sapiens
FPVEEQQEVPPETNRKTDDPEQKAKVKKKK

SEQ ID NO: 37 - protein, 758 aa, Homo Sapiens [sequence omitted]

SEQ ID NO: 38 - protein, 414 aa, Homo Sapiens
LDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDPGTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETRTWEEGKVLIFDDSFEHEVWQDASSFRLIFIVDVWHPELTPQQRRSLPAI

SEQ ID NO: 39 - protein, 28 aa, Homo Sapiens
ASSFRLIFIVDVWHPELTPQQRRSLPAI

SEQ ID NO: 40 - protein, 78 aa, Homo Sapiens
GLSGTSFFTWFMVIALLGVWTSVAGGSGGGFAKVHYGFILKAQNKIAESIPGGSGGHTGPTNCRLRMHLGLVIPKEGC

SEQ ID NO: 41 - protein, 85 aa, Homo Sapiens
GLSGTSFFTWFMVIALLGVWTSVAGGSGGIEEAVNAFKELVRKYPQSPRARYGKAQCGGSGGDVPADLLKLSLKRRSDRQQFLGH

SEQ ID NO: 42 - protein, 51 aa, Homo Sapiens
DVPADLLKLSLKRRSDRQQFLGHGGSGGSLERNWKLIRDEGLAVMDKAKGL

SEQ ID NO: 43 - protein, 51 aa, Homo Sapiens; identical to SEQ ID NO: 19

SEQ ID NO: 44 - protein, 10 aa, Homo Sapiens
CYPYDVPDYA

SEQ ID NO: 45 - protein, 8 aa, Homo Sapiens
DYKDDDDK

SEQ ID NO: 46 - protein, 11 aa, Homo Sapiens
CEQKLISEEDL

SEQ ID NO: 47 - protein, 6 aa, Homo Sapiens
HHHHHH

SEQ ID NO: 48 - protein, 10 aa, Homo Sapiens
EQKLISEEDL

SEQ ID NO: 49 - protein, 8 aa, Homo Sapiens
DYKDDDDK

SEQ ID NO: 50 - protein, 8 aa, Homo Sapiens
WSHPQFEK

SEQ ID NO: 51 - protein, 9 aa, Homo Sapiens
YPYDVPDYA

SEQ ID NO: 52 - protein, 5 aa, Homo Sapiens
RYIRS

SEQ ID NO: 53 - protein, 17 aa, Homo Sapiens
WEAAAREACCRECCARA

(19) United States
(12) Patent Application Publication
(10) Pub. No.: US 2020/0190480 A1
(43) Pub. Date: Jun. 18, 2020

(54) ANTIBODIES AGAINST ASPARTYL (ASPARAGINYL) beta-HYDROXYLASE (AABH) AND METHODS OF MAKING AND USING

(71) Applicant: Next Pharma Inc., Richmond Hill (CA)

(72) Inventors: Mahmood MOSHIRI, Richmond Hill, Ontario (CA); Kiarash MOSHIRI, Richmond Hill, Ontario (CA); Arsha MOSHIRI, Richmond Hill, Ontario (CA); Mohammad Hadi SEKHAVATI, Mashhad, Khorasan (IR); Hassan MONHEMI, Mashhad, Khorasan (IR)

(21) Appl. No.: 16/643,509

(22) PCT Filed: Aug. 28, 2018
(86) PCT No.: PCT/CA2018/051041

§ 371 Date: Feb. 28, 2020

Related U.S. Application Data
(60) Provisional application No. 62/551,855, filed on Aug. 30, 2017.

(51) Int. Cl. C12N 5/16; C07K 16/40; C12N 9/02; A61K 47/68; C12N 15/85; A61K 39/00; G01N 33/574; A61K 38/44; A61K 45/06

(52) U.S. Cl. CPC C12N 5/163; C07K 16/40; C12N 9/0071; C12Y 114/11016; A61K 45/06; C12N 15/85; A61K 39/0011; G01N 33/57492; A61K 38/44; A61K 47/6871

(57) ABSTRACT

Provided herein are polypeptide epitopes of aspartyl-(asparaginyl)-3-hydroxylase (“AABH”) as well as polyepitopes thereof. Also provided are antibodies that specifically bind these polypeptide epitopes and poly epitopes, as well as binding the aspartyl-(asparaginyl)-3-hydroxylase protein itself. The disclosure further provides methods of assaying for AABH polypeptide epitopes, cells expressing these polypeptide epitopes and the AABH protein. Also provided are methods of diagnosing cancer by the detection of AABH peptides and methods of treating cancer by targeting cells that express AABH.

### REFERENCE TO RELATED APPLICATIONS

This application claims the benefit of U.S. Provisional application No. 62/551,855 filed Aug. 30, 2017 which is incorporated herein by reference in its entirety.

### STATEMENT AS TO FEDERALLY SPONSORED RESEARCH

None.

### BACKGROUND

Aspartyl-(asparaginyl)-β-hydroxylase is a member of the α-ketoglutarate-dependent dioxygenase family that catalyzes the hydroxylation of aspartyl and asparaginyl residues in epidermal growth factor (EGF)-like domains of protein. Aspartyl-(asparaginyl)-β-hydroxylase is variously referred to herein as “AABH,” “AAH,” ASPH″ and “HAAH”. In humans, it is encoded by the ASPH gene.

EGF-like domains are present in proteins involved in cellular signaling pathways and proteins that interact with the extracellular matrix. Both of these types of proteins have been demonstrated to play important roles in the development and maintenance of cancer. In particular, AABH functions as a hydroxylating enzyme that promotes cell motility by enhancing Notch-Jagged-HES-1 signaling. This signaling pathway is known to affect cell fate decisions and growth control during cellular maturation. Notch has 17 EGF-like domains and is involved in cancer phenotypes such as proliferation, motility and invasiveness affecting tumor growth and metastatic potential. A direct interaction between Notch and AABH has been demonstrated in tumor cells.

AABH is normally localized to the endoplasmic reticulum. In malignant cells, it is expressed as a transmembrane enzyme. AABH has been shown to be expressed at higher levels in malignant cells than in adjacent non-malignant cells in a variety of cancers, including lung, liver, colon, pancreas, prostate, ovary, bile duct, and breast.

In addition, AABH is thought to play a significant role in calcium homeostasis.

U.S. Patent Application Publication 2003/0031670 (Wands et al.) refers to a method for diagnosing a malignant neoplasm in a mammal by contacting a bodily fluid from the mammal with an antibody which binds to a human aspartyl (asparaginyl) β-hydroxylase polypeptide.

U.S. Patent Application Publication 2015/0306198 (Wands et al.) refers to a peptide-based immunotherapy for ASPH-expressing tumors.

U.S. Patent Application Publication 2016/0354499 (Ghanbari et al.) refers to methods for making and using radio-labeled anti-HAAH antibodies for tumor imaging and immunotherapy.

### SUMMARY

In one aspect, provided herein is a polypeptide having an amino acid sequence consisting of, or consisting essentially of: SEQ ID NO: 1—CRRGQIKYS; SEQ ID NO: 2-GPTNCRLRMHLGLVI; SEQ ID NO: 3—RT WEEGKVLIFD; SEQ ID NO: 4—WQD ASSFRLIFI; SEQ ID NO: 5-LS GTSFFTWFMVIALLGVWTSV; SEQ ID NO: 6—VYEEVLSVTPNDGFAKVHYGFILKAQNK; SEQ ID NO: 7-FASVWQRSLYNVNGLK; SEQ ID NO: 8—GLSGTSFFTWFMVIALLGVWTSVA; SEQ ID NO: 9-GDGDFDVDDAKVLLGLKERSTSEPAV; SEQ ID NO: 10—IEEAVNAFKELVRKYPQSPRARYGKAQC; SEQ ID NO: 11—DVPADLLKLSLKRRSDRQQFLGH; SEQ ID NO: 12—SLERNWKLIRDEGLAVMDKAKGL; SEQ ID NO: 13—GFAKVHYGFILKAQNKIAESIP; SEQ ID NO: 14—HTGPINCRLRMHLGLVIPKEGC; SEQ ID NO: 15-DSFEHEVWQDASSFRLIFIVDVW; SEQ ID NO: 16—MRGSLLTLQRLVQLFPNDTSLKN; SEQ ID NO: 17-PQQEDDEFLMATDVDDRFETL; SEQ ID NO: 18—DGRFYFHLGDAMQRVGNKEAY; SEQ ID NO: 19-GLSGTSFFTW FMVIALLGVW TSVAGGSGGG FAKVHYGFIL KAQNKIAESIP; SEQ ID NO: 20—KVYEEVLSVE EVLSVTPNTP NDGFAKVGFA KVHYGFKVHY GFILKKIAES IPYL; SEQ ID NO: 21-GTDDGRFYFRVGNKEAYKASVWQRSLYSLYNVNGLK; SEQ ID NO: 22—SFFTWFMVIALLGVWTSVA; SEQ ID NO: 23—PADLLKLSLKRRSDRQQF; SEQ ID NO: 24—GFAKVHYGFILKAQNKIAESIPY; SEQ ID NO: 25-TGPTNCRLRMHLGLVIPKEGC; SEQ ID NO: 26—FEHEVWQDASSFRLIFVDVWHPEL; SEQ ID NO: 27-EHVEGEDLQQEDGPTGEPQQEDDEFL; SEQ ID NO: 28—PYLKEGIESGDPGTDDGR; SEQ ID NO: 29-GLKAQPWWTPKETGYTE; SEQ ID NO: 30—LKAQNKIAESIPYLKEGI; SEQ ID NO: 31-HLGDAMQRVGNKEAYKWYELGHKRGHFASVW; SEQ ID NO: 32—VDVWHPELTPQQRRSLPAI; SEQ ID NO: 33—KNAKSSGNSSSSGSGSGSTSAGSSSPGARRE; SEQ ID NO: 34-IYDADGDGDFDVDDAKVLLGLKERSTSEPAVP; SEQ ID NO: 35—EEMM SEQENPDSSEPVVE; and SEQ ID NO: 36—FPVEEQQEVPPETNRKTDDPEQKAKVKKKK. In one embodiment, the polypeptide is in substantially pure form.

In another aspect, provided herein is polypeptide having an amino acid sequence consisting of, or consisting essentially of AABH Fragment 1 (SEQ ID NO:38) or AABH Fragment 2 (SEQ ID NO:39).

In another aspect, provided herein is a polypeptide having an amino acid sequence consisting of a fragment of a polypeptide of SEQ ID NO: 1-39, wherein the fragment is no more than 1, 2, 3 or 4 amino acids shorter than the polypeptide of claim 1. In certain embodiments, the polypeptide is no more than 25, no more than 35, no more than 45 or no more than 55 amino acids long.

In another aspect, provided herein is a chimeric polyepitope comprising: 2, 3 or 4 polypeptides having an amino acid sequence consisting of, or consisting essentially of: SEQ ID NO: 1—CRRGQIKYS; SEQ ID NO: 2—GPTNCRLRMHLGLVI; SEQ ID NO: 3—RT WEEGKVLIFD; SEQ ID NO: 4-WQD ASSFRLIFI; SEQ ID NO: 5—LS GTSFFTWFMVIALLGVWTSV; SEQ ID NO: 6—VYEEVLSVTP NDGFAKVHYG FILKAQNK; SEQ ID NO: 7—FASVWQRSLYNVNGLK; SEQ ID NO: 8-GLSGTSFFTWFMVIALLGVWTSVA; SEQ ID NO: 9—GDGDFDVDDAKVLLGLKERSTSEPAV; SEQ ID NO: 10-IEEAVNAFKELVRKYPQSPRARYGKAQC; SEQ ID NO: 11—DVPADLLKLSLKRRSDRQQFLGH; SEQ ID NO: 12—SLERNWKLIRDEGLAVMDKAKGL; SEQ ID NO: 13—GFAKVHYGFILKAQNKIAESIP; SEQ ID NO: 14-HTGPINCRLRMHLGLVIPKEGC; SEQ ID NO: 15—DSFEHEVWQDASSFRLIFIVDVW; SEQ ID NO: 16-MRGSLLTLQRLVQLFPNDTSLKN; SEQ ID NO: 17—PQQEDDEFLMATDVDDRFETL; SEQ ID NO: 18-DGRFYFHLGDAMQRVGNKEAY; SEQ ID NO: 19-GLSGTSFFTWFMVIALLGVWTSVAGGSGGGFAKVHYGFILKAQNKIAESIP; SEQ ID NO: 20—KVYEEVLSVE EVLSVTPNTP NDGFAKVGFA KVHYGFKVHY GFILKKIAES IPYL; SEQ ID NO: 21—GTDDGRFYFR VGNKEAYKAS VWQRSLYSLYNVNGLK; SEQ ID NO: 22—SFFTWFMVIALLGVWTSVA; SEQ ID NO: 23-PADLLKLSLKRRSDRQQF; SEQ ID NO: 24—GFAKVHYGFILKAQNKIAESIPY; SEQ ID NO: 25-TGPTNCRLRMHLGLVIPKEGC; SEQ ID NO: 26—FEHEVWQDASSFRLIFVDVWHPEL; SEQ ID NO: 27-EHVEGEDLQQEDGPTGEPQQEDDEFL; SEQ ID NO: 28—PYLKEGIESGDPGTDDGR; SEQ ID NO: 29-GLKAQPWWTPKETGYTE; SEQ ID NO: 30—LKAQNKIAESIPYLKEGI; SEQ ID NO: 31-HLGDAMQRVGNKEAYKWYELGHKRGHFASVW; SEQ ID NO: 32—VDVWHPELTPQQRRSLPAI; SEQ ID NO: 33—KNAKSSGNSSSSGSGSGSTSAGSSSPGARRE; SEQ ID NO: 34-IYDADGDGDFDVDDAKVLLGLKERSTSEPAVP; SEQ ID NO: 35—EEMM SEQENPDSSEPVVE; SEQ ID NO: 36—FPVEEQQEVPPETNRKTDDPEQKAKVKKKK; SEQ ID NO: 38 or SEQ ID NO: 39 wherein the polypeptides are covalently connected through one or more linkers.

In one embodiment, the chimeric polyepitope consists of, or consists essentially of an amino acid sequence selected from: SEQ ID NO: 40 GLSGTSFFTW FMVIALLGVW TSVAGGSGGG FAKVHYGFIL KAQNKIAESI PGGSGGHTGP TNCRLRMHLG LVIPKEGC; SEQ ID NO: 41 LSGTSFFTWF MVIALLGVWT SVAGGSGGIE EAVNAFKELV RKYPQSPRARYGKAQCGGSGGDVPADLLKLSLKRRSDRQQFLGH; SEQ ID NO: 42 DVPADLLKLSLKRRSDRQQFLGHGGSGGSLERNWKLIRDEGLAVMDKAKGL; SEQ ID NO: 43 GLSGTSFFTWFMVIALLGVWTSVAGGSGGGFAKVHYGFILKAQNKIAESIP.

In another aspect, provided herein is a monoclonal antibody that specifically binds to a polypeptide is selected from: (i) a polypeptide having an amino acid sequence consisting of, or consisting essentially of a polypeptide selected from SEQ ID NO:1-SEQ ID NO:36; (ii) a polypeptide having an amino acid sequence consisting of a fragment of a polypeptide selected from SEQ ID NO: 1-SEQ ID NO: 36, wherein the fragment is no more than 1, 2, 3 or 4 amino acids shorter than the polypeptide selected from SEQ ID NO: 1-SEQ ID NO: 36; (iii) a chimeric polyepitope comprising 2, 3 or 4 polypeptides having an amino acid sequence consisting of, or consisting essentially of a polypeptide selected from SEQ ID NO: 1-SEQ ID NO: 36, wherein the polypeptides are covalently connected through one or more linkers; and (iv) a polypeptide having an amino acid sequence consisting of, or consisting essentially of SEQ ID NO: 38—AABH Fragment 1 or SEQ ID NO: 39—AABH Fragment 2. In one embodiment, the monoclonal antibody binds to aspartyl (asparaginyl) β-hydroxylase (“AABH”). In another embodiment, the monoclonal antibody is a humanized monoclonal antibody. In another embodiment, the monoclonal antibody is monospecific. In another embodiment, the monoclonal antibody is multispecific or bispecific. In another embodiment, the monoclonal antibody is a whole immunoglobulin. In another embodiment, the monoclonal antibody is an antibody fragment, e.g., selected from the group consisting of: Fab, F(ab′)2, Fab′ or single chain Fv.

In another aspect, provided herein is a conjugated monoclonal antibody comprising a monoclonal antibody as disclosed herein, conjugated with a chemical moiety. In one embodiment, the chemical moiety is a detectable label. In another embodiment, the chemical moiety is chemotherapeutic or cytotoxic agent. In another embodiment, the chemotherapeutic or cytotoxic agent is selected from the group consisting of: alkylating agent, anti-metabolite, antibiotic, hydroxyurea, platinum-based chemotherapeutic agent, taxane, bortezomib, lenalidomine, thalidomide and metanzinoid.

In another aspect, provided herein is a composition comprising an adjuvant and a monoclonal antibody or conjugated monoclonal antibody as disclosed herein. In one embodiment, the composition is pharmaceutically acceptable.

In another aspect, provided herein is an article comprising a monoclonal antibody or conjugated monoclonal antibody as disclosed herein affixed to a solid support. In one embodiment, the solid support is selected from the group consisting of a microtiter plate, an assay plate, an assay well, a nitrocellulose membrane, a bead, a dipstick, and a component of an elution column.

In another aspect, provided herein is a kit comprising: a) a container comprising a first monoclonal antibody or conjugated monoclonal antibody as disclosed herein, and b) one or more of: (i) a container comprising a second, different monoclonal antibody or conjugated monoclonal antibody as disclosed herein, (ii) an assay plate with or without antibody attached, and (iii) reagents for performing an immunoassay. In one embodiment, the kit comprises: (i) a container comprising a second, different monoclonal antibody or conjugated monoclonal antibody as disclosed herein. In another embodiment the second monoclonal antibody or conjugated monoclonal antibody comprises a detectable label. In another embodiment, the kit comprises: (ii) an assay plate with or without antibody attached. In another embodiment, the kit comprises: (iii) reagents for performing an immunoassay.

In another aspect, provided herein is a complex comprising a monoclonal antibody as disclosed herein, bound to AABH, a polypeptide of SEQ ID NO: 1-39, chimeric polyepitope as disclosed herein or a polypeptide comprising an amino acid sequence selected from any one or more of SEQ ID NOs. 1-39. In one embodiment, the polypeptide comprises a plurality of amino acid sequences selected from the group consisting of SEQ ID NOs. 1-36. In another embodiment, polypeptide is aspartyl (asparaginyl) β-hydroxylase (“AABH”).

In another aspect provided herein is a method of making a hybridoma that produces antibodies against aspartyl (asparaginyl) β-hydroxylase (“AABH”) comprising: a) immunizing an animal with a polypeptide to elicit an immune response, wherein the polypeptide is selected from: (i) a polypeptide having an amino acid sequence consisting of, or consisting essentially of a polypeptide selected from SEQ ID NO:1-SEQ ID NO:36; (ii) a polypeptide having an amino acid sequence consisting of a fragment of a polypeptide selected from SEQ ID NO: 1-SEQ ID NO: 36, wherein the fragment is no more than 1, 2, 3 or 4 amino acids shorter than the polypeptide selected from SEQ ID NO: 1-SEQ ID NO: 36; (iii) a chimeric polyepitope comprising 2, 3 or 4 polypeptides having an amino acid sequence consisting of, or consisting essentially of a polypeptide selected from SEQ ID NO: 1-SEQ ID NO: 36, wherein the polypeptides are covalently connected through one or more linkers; and (iv) a polypeptide having an amino acid sequence consisting of, or consisting essentially of SEQ ID NO: 38—AABH Fragment 1 or SEQ ID NO: 39—AABH Fragment 2; b) isolating B cells from the animal; c) fusing the isolated B cells with HAT-sensitive myeloma cells to produce hybridoma cells; and d) selecting HAT (hypoxanthine-aminopterin-thymidine) tolerant hybridoma cells that produce antibodies that bind to the polypeptide. In one embodiment, immunizing comprises injecting the animal with the polypeptide a plurality of times over a plurality of weeks. In another embodiment the B cells are isolated from the animal's spleen. In another embodiment immunizing comprises in vivo electroporation of the polypeptide. In another embodiment fusing comprises use of an electric field or exposure to polyethylene glycol. In another embodiment the method further comprises: e) cloning the selected hybridoma cells. In another embodiment the method further comprises e) isolating monoclonal antibodies from the selected hybridoma cells.

In another aspect, provided herein is a method of eliciting antibodies in a mammalian subject comprising administering to the subject a polypeptide or a chimeric polyepitope as disclosed herein. In one embodiment, the mammalian subject is selected from mouse, rat, goat, sheep, primate, or human.

In another aspect, provided herein is a method of making a hybridoma that produces antibodies that bind to a polypeptide as disclosed herein.

In another aspect, provided herein is a hybridoma that produces a monoclonal antibody as disclosed herein.

In another aspect, provided herein is a nucleic acid molecule comprising a nucleotide sequence encoding an immunoglobulin polypeptide of a monoclonal antibody as disclosed herein.

In one embodiment, the nucleic acid molecule comprises an expression construct comprising an expression control sequence operably linked to the nucleotide sequence. In another embodiment, the nucleic acid molecule is comprised in an expression vector.

In another aspect, provided herein is a host cell comprising one or more expression constructs, each expression construct comprising and expression control sequence operatively linked with a nucleotide sequence encoding an immunoglobulin chain of a monoclonal antibody as disclosed herein. In one embodiment, the host cell is selected from the group consisting of a fungal cell, an insect cell and a mammalian cell. In another embodiment of the host cell a Chinese Hamster Ovary (CHO) cell.

In another aspect, provided herein is a process for creating a monoclonal antibody comprising culturing a hybridoma producing a monoclonal antibody or a host cell as described herein.

In another aspect, provided herein is a method comprising detecting aspartyl (asparaginyl) β-hydroxylase (“AABH”) with an immunoassay that uses a monoclonal antibody as disclosed herein. In one embodiment, the immunoassay is an ELISA assay, a radioimmunoassay or a sandwich immunoassay.

In another aspect, provided herein is a method of detecting aspartyl (asparaginyl) β-hydroxylase (“AABH”) in a sample comprising contacting the sample with a monoclonal antibody as disclosed herein, and detecting binding of the antibody to AABH.

In another aspect, provided herein is a method of diagnosing cancer in a subject comprising contacting a biological sample from the subject with a monoclonal antibody as disclosed herein and determining a quantitative measure of an amount of aspartyl (asparaginyl) β-hydroxylase (“AABH”) in the sample, wherein an amount of AABH above a cut off level indicates cancer. In one embodiment, the cutoff level is 3.6. ng/ml serum. In another embodiment, the biological sample is selected from blood, serum, plasma, cerebrospinal fluid (CSF), solid tissue, or biopsy. In another embodiment, the cancer is selected from lung, liver, colon, pancreas, prostate, ovary, bile duct, and breast. In another embodiment, the cancer is not detectable in the subject by imaging methods, e.g., selected from PET scan, MRI, X-ray, CAT-Scan and ultrasound. In another embodiment, the method is capable of detecting one cancer cell in a background of 50,000 normal cells. In another embodiment the method further comprises: administering a therapeutic intervention to the subject diagnosed with cancer.

In another aspect, provided herein is a method of diagnosing cancer in a subject, the method comprising: (a) providing a biological sample from the subject; (b) contacting the biological sample with a monoclonal antibody as disclosed herein; (c) measuring an amount of AABH in the sample based binding of the antibody to AABH; (d) determining of the measure is greater than a cut off value, wherein a measure greater than the cut off value provides a diagnosis of cancer, and wherein the cut off value is based on measurements of AABH in a plurality of cancer positive samples and a plurality of cancer negative samples. In one embodiment, the plurality of positive samples and the plurality of negative samples is each at least 10, at least 20, at least 50 or at least 100.

In another aspect, provided herein is a method for treating cancer in a subject in need thereof, the method comprising administering to the subject, a monoclonal antibody or conjugated monoclonal antibody as disclosed herein, or a pharmaceutical composition as disclosed herein. In a further aspect, provided herein is a use of a monoclonal antibody or conjugated monoclonal antibody or a pharmaceutical composition as disclosed herein for treating cancer in a subject in need thereof. In yet a further aspect, provided herein is a use of a monoclonal antibody or conjugated monoclonal antibody or a pharmaceutical composition as disclosed herein in the manufacture of a medicament for treating cancer in a subject in need thereof. In another aspect, provided herein is a monoclonal antibody or conjugated monoclonal antibody or a pharmaceutical composition as disclosed herein for use in treating cancer. In one embodiment, treatment comprises inhibiting metastasis.

In another aspect, provided herein is a method for immunizing a subject against AABH comprising administering to the subject, one or more of the polypeptides or chimeric polyepitopes as disclosed herein. In one embodiment, the administration is parenteral. In another embodiment, the administration is intravenous.

In another aspect, provided herein is a nucleic acid encoding a polypeptide or chimeric polyepitope as disclosed herein.

In another aspect, provided herein is an expression construct comprising an expression control sequence operatively linked with a nucleotide sequence encoding a polypeptide or chimeric polyepitope as disclosed herein.

In another aspect, provided herein is a host cell comprising the expression construct as disclosed herein. In one embodiment, the cell is a mammalian cell, e.g., a CHO cell.

In another aspect, provided herein is a vaccine comprising one or a plurality of the polypeptides or chimeric polyepitopes as disclosed herein.

In another aspect, provided herein is a method for determining the presence of a malignant cell in a tissue section, the method comprising: (a) contacting the section with a monoclonal antibody or conjugated monoclonal antibody as disclosed herein; and (b) testing for binding of the antibody to the surface of cells in the tissue section; wherein a cell whose surface is bound by the antibody is malignant.

In another aspect, provided herein is a method for determining the presence of a malignant cell in a subject, the method comprising: (a) administering to the subject a monoclonal antibody as disclosed herein conjugated with a detectable moiety; and (b) detecting binding of the antibody to a cell in the subject by an imaging method; wherein detection of binding a cell indicates that the cell is malignant.

In another aspect, provided herein is a method comprising: (a) administering to a subject determined to have a level of AABH above a diagnostic cut off for cancer, a therapeutic intervention for the cancer. In one embodiment the level is determined from a blood sample from the subject. In another embodiment the diagnostic cut off is at least 0.25 ng/ml, 0.3 ng/ml or 0.3 ng/ml. In another embodiment the diagnostic cut off has a specificity of at least 90%, at least 95%, at least 99% or at least 99.5%. In another embodiment the diagnostic cut off has a sensitivity of at least 90%, at least 95%, at least 99% or at least 99.5%. In another embodiment the diagnostic cut off has a precision of at least 90%, at least 95%, at least 99% or at least 99.5%. In another embodiment the therapeutic intervention comprises administration of chemotherapy, radiation therapy or immunotherapy.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIGS. 1A-1D show measurements of AABH in breast, colon, lung and prostate cancers, and a cut off of about 2 ng/ml for a diagnosis of cancer. FIG. 1A shows measurements of AABH in breast cancer patients and non-cancer patients. Using the cut off shown, the test inferred cancer with 70% accuracy. FIG. 1B shows measurements of AABH in colon cancer patients and non-cancer patients. Using the cut off shown, the test inferred cancer with 91.5% accuracy. FIG. 1C shows measurements of AABH in lung cancer patients and non-cancer patients. Using the cut off shown, the test inferred cancer with 93% accuracy. FIG. 1D shows measurements of AABH in prostate cancer patients and non-cancer patients. Using the cut off shown, the test inferred cancer with 91% accuracy.

FIG. 2 shows an exemplary an exemplary polyepitope of this disclosure.

FIG. 3 shows results of a test measuring AABH in control and test samples.

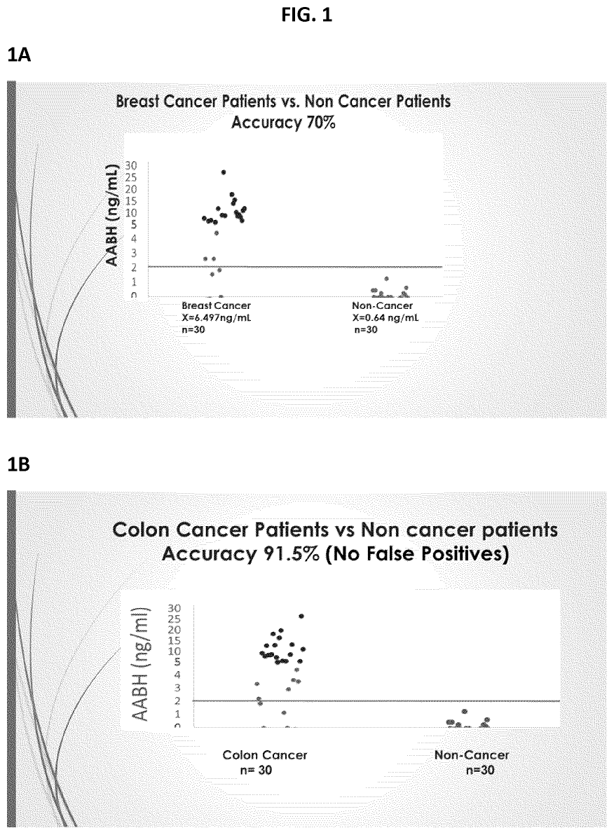

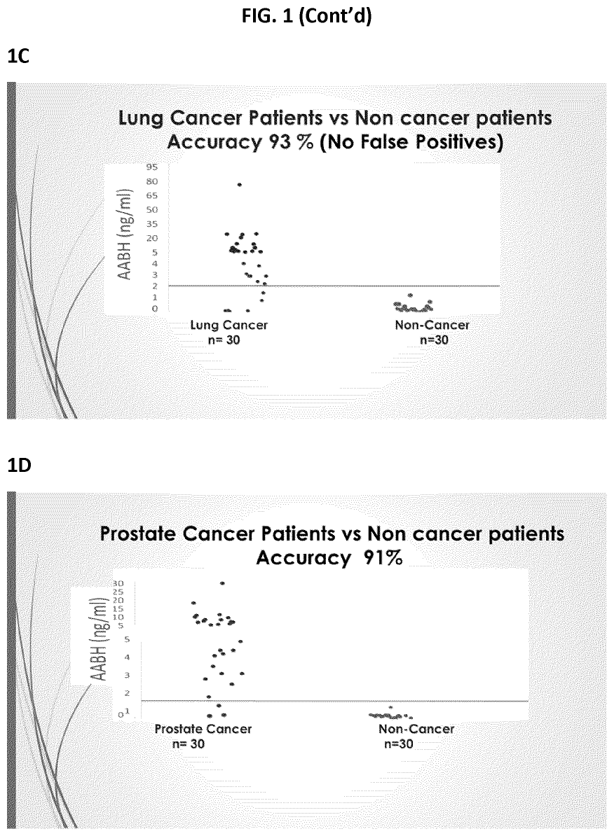

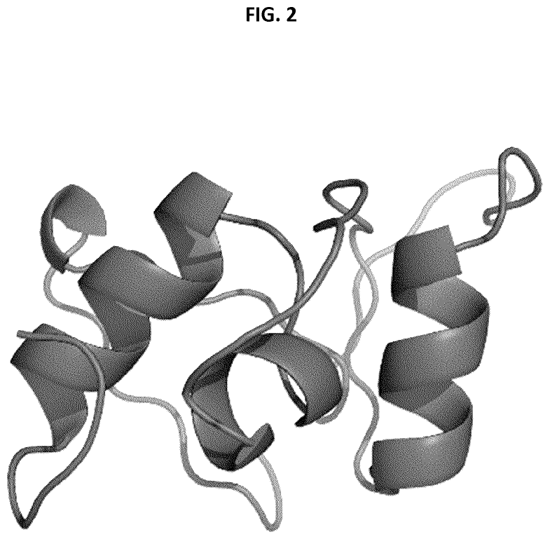

### DETAILED DESCRIPTION

### I. Definitions

“Aspartyl (asparaginyl) β-hydroxylase” (“AABH”) refers to a protein having an amino acid sequence of SEQ ID NO:37. AABH also refers to allelic forms of this protein in humans as well as all mammalian cognate and alleles thereof. The amino acid sequence of human AABH is:

MAQRKNAKSS GNSSSSGSGS GSTSAGSSSP GARRETKHGG
HKNGRKGGLS GTSFFTWFMV IALLGVWTSV AVVWFDLVDY
EEVLGKLGIY DADGDGDFDV DDAKVLLGLK ERSTSEPAVP
PEEAEPHTEP EEQVPVEAEP QNIEDEAKEQ IQSLLHEMVH
AEHVEGEDLQ QEDGPTGEPQ QEDDEFLMAT DVDDRFETLE
PEVSHEETEH SYHVEETVSQ DCNQDMEEMM SEQENPDSSE
PVVEDERLHH DTDDVTYQVY EEQAVYEPLE NEGIEITEVT
APPEDNPVED SQVIVEEVSI FPVEEQQEVP PETNRKTDDP
EQKAKVKKKK PKLLNKFDKT IKAELDAAEK LRKRGKIEEA
VNAFKELVRK YPQSPRARYG KAQCEDDLAE KRRSNEVLRG
AIETYQEVAS LPDVPADLLK LSLKRRSDRQ QFLGHMRGSL
LTLQRLVQLF PNDTSLKNDL GVGYLLIGDN DNAKKVYEEV
LSVTPNDGFA KVHYGFILKA QNKIAESIPY LKEGIESGDP
GTDDGRFYFH LGDAMQRVGN KEAYKWYELG HKRGHFASVW
Q RSLYNVNGL KAQPWWTPKE TGYTELVKSL ERNWKLIRDE
GLAVMDKAKG LFLPEDENLR EKGDWSQFTL WQQGRRNENA
CKGAPKTCTL LEKFPETTGC RRGQIKYSIM HPGTHVWPHT
GPTNCRLRMH LGLVIPKEGC KIRCANETRT WEEGKVLIFD
DSFEHEVWQD ASSFRLIFIV DVWHPELTPQ QRRSLPAI

(SEQ ID NO:37. GENBANK Accession No. S83325; His motif is underlined; conserved sequences within the catalytic domain are designated by bold type.)

As used herein, the term “polypeptide” refers to a molecule having a sequence of natural and/or unnatural amino acids connected through peptide bonds. The term “peptide” refers to a short polypeptide, typically no more than 30 amino acids long. The amino acid sequence of a polypeptide is referred to as its “primary structure.” The term “protein” refers to a polypeptide having a secondary, tertiary and/or quaternary structure, e.g., structures stabilized by hydrogen bonds, relationships between secondary structures and structures formed of more than one protein. Proteins can be further modified by other attached moieties such as carbohydrate (glycoproteins), lipids (lipoproteins) phosphate groups (phosphoproteins) and the like.

As used herein, an amino acid sequence “consists of” only the amino acids in that sequence.

As used herein, a first amino acid sequence “consists essentially of” a second amino acid sequence if the first amino acid sequence (1) comprises the second amino sequence and (2) is no more than 1, no more than 2 or no more than 3 amino acids longer than the second amino acid sequence.

As used herein, a first amino acid sequence is a “fragment” of a second amino acid sequence if the second amino acid sequence comprises the first amino acid sequence. In certain embodiments, a first amino acid sequence that is a fragment of a second amino acid sequence may have no more than any of 1, 2, 3, 4, 5, 6, 7, 8, 9 or 10 fewer amino acids than the second amino acid sequence.

As used herein, a “functional equivalent” of a reference amino acid sequence is a sequence that is not identical to the reference sequence, but that contains minor alterations such as, for example, insertion, deletion or substitution of one or a few amino acids. A functionally equivalent sequence retains the function (e.g., immunogenicity) of the reference sequence to which it is equivalent. If a functionally equivalent amino acid sequence contains substitution of one or more amino acids with respect to the reference sequence, these will generally be conservative amino acid substitutions.

The term “conservative amino acid substitution” refers substituting a first amino acid with a second amino acid in a grouping amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). A group of amino acids containing aromatic side chains includes phenylalanine, tryptophan and tyrosine. Heterocyclic side chains are present in proline, tryptophan and histidine. Within the group of amino acids containing non-polar side chains, those with short hydrocarbon side chains (glycine, alanine, valine. leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

Another basis for determining functional equivalence of amino acids in peptides is based on analyses of amino acid changes between homologous proteins from different organisms. According to such analyses, groups of amino acids can be defined in which amino acids within a group are preferentially substituted for one another in homologous proteins, and therefore have similar impact on overall protein structure (Schulz, G. E. and R. H. Schirmer, Principles of Protein Structure, Springer-Verlag, 1979). According to this type of analysis, the following groups of amino acids can be conservatively substituted for one another:

(i) amino acids containing a charged group, consisting of Glu, Asp, Lys, Arg and His;

(ii) amino acids containing a positively-charged group, consisting of Lys, Arg and His;

(iii) amino acids containing a negatively-charged group, consisting of Glu and Asp;

(iv) amino acids containing an aromatic group, consisting of Phe, Tyr and Trp;

(v) amino acids containing a nitrogen ring group, consisting of His and Trp;

(vi) amino acids containing a large aliphatic non-polar group, consisting of Val, Leu and Ile;

(vii) amino acids containing a slightly-polar group, consisting of Met and Cys;

(viii) amino acids containing a small-residue group, consisting of Ser, Thr, Asp, Asn, Gly, Ala, Glu, Gln and Pro;

(ix) amino acids containing an aliphatic group consisting of Val, Leu, Ile, Met and Cys; and

(x) amino acids containing a hydroxyl group consisting of Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, Calif., p. 224.

The term “substantially identical” refers to identity between a first amino acid sequence that contains a sufficient or minimum number of amino acid residues that are i) identical to, or ii) conservative substitutions of aligned amino acid residues in a second amino acid sequence such that the first and second amino acid sequences have a common structural domain and/or common functional activity and/or common immunogenicity. For example, amino acid sequences that contain a common structural or antigenic domain having at least about 90%, 91%, 92%, 93%, 94%, 95%, 96%, 97%, 98% or 99% identity are termed sufficiently or substantially identical. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, or encode polypeptides having the same immunogenic properties.

As used herein, the term “chimeric polyepitope” refers to a plurality of polypeptides connected to each other through linkers, e.g., peptide linkers. See, for example, Chen et al. (2013) Adv. Drug Deliv. Rev. 65:1357-1369. Additional linkers include, without limitation, polyethylene glycol (PEG), β-alanine, 4-aminobutyric acid (GABA), (2-aminoethoxy)acetic acid (AEA), 5-aminovaleric acid (AVA), 6-aminocaproic acid (Ahx), 8-amino-3,6-dioxaoctanoic acid (mini-PEG1), 12-amino-4,7,10-trioxadodecanoic acid (mini-PEG2), 15-amino-4,7,10,13-tetraoxapentadecanoic acid (mini-PEG3) and trioxatridecansuccinamic acid.

As used herein, a chemical entity, such as a polypeptide, is “substantially pure” if it is the predominant chemical entity of its kind (e.g., of polypeptides) in a composition. This includes the chemical entity representing more than 50%, more than 80%, more than 90%, more than 95%, more than 98%, more than 99%, more than 99.5%, more than 99.9%, or more than 99.99% of the chemical entities of its kind in the composition.

As used herein, the terms “antigen,” “immunogen,” and “antibody target,” refer to a molecule, compound, or complex that is recognized by an antibody, i.e., can be bound by the antibody. The term can refer to any molecule that can be recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein the term “antibody” refers to a polypeptide comprising a framework region from an immunoglobulin gene, or fragments thereof (“antibody fragment”), that binds (e.g., “recognize”) an antigen or an epitope. Immunoglobulins typically comprise a “variable region” and a “constant region”. The variable region contains the antigen-binding region of the antibody (or its functional equivalent) and is most critical in specificity and affinity of binding. An exemplary immunoglobulin (antibody) structural unit comprises a tetramer. Each tetramer is composed of two identical pairs of polypeptide chains, each pair having one “light chain” (about 25 kD) and one “heavy chain” (about 50-70 kD).

An “isotype” is a class of antibody defined by the heavy chain constant region. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively.

Antibodies can exist as intact immunoglobulins or as any of a number of well-characterized fragments that possess specific antigen-binding activity. For the sake of clarity, a tetrameric antibody with full-length heavy and light chains is referred to herein as an “intact immunoglobulin,” and can be naturally occurring, polyclonal, monoclonal, or recombinantly produced. Fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of products of the digestion of an intact antibody, one of skill will appreciate that such fragments may also be synthesized de novo chemically or constructed and expressed using recombinant DNA methodology. Thus, the term “antibody”, as used herein, also includes antibody fragments either produced by the modification of whole antibodies, or those synthesized de novo using chemical methods, or those created and expressed by recombinant DNA methodologies or those identified using phage display libraries (see, e.g., McCafferty et al., Nature 348:552-554 (1990)).

As used herein, the term “Fv” refers to a monovalent or bi-valent variable region fragment, and can encompass only the variable regions (e.g., V_{L} and/or V_{H}), as well as longer fragments, e.g., an Fab, Fab′ or F(ab′)2, which also includes C_{L} and/or C_{H}1. Unless otherwise specified, the term “Fc” refers to a heavy chain monomer or dimer comprising C_{H}1 and C_{H}2 regions.

A single chain Fv (scFv) refers to a polypeptide comprising a V_{L} and V_{H} joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

Antibody fragments further include Fd (the portion of the heavy chain included in the Fab fragment) and single domain antibodies. A single domain antibody (sdAb) is a variable domain of either a heavy chain or a light chain, produced by recombinant methods.

The specificity of the binding can be defined in terms of the comparative dissociation constants (Kd) of the antibody (or other targeting moiety) for target, as compared to the dissociation constant with respect to the antibody and other materials in the environment or unrelated molecules in general. A larger (higher) K_{d} is a K_{d} that describes a lower affinity interaction. Conversely a smaller (lower) K_{d} is a K_{d} that describes a higher affinity interaction or tighter binding. By way of example only, the K_{d} for an antibody specifically binding to a target may be femtomolar, picomolar, nanomolar, or micromolar and the K_{d} for the antibody binding to unrelated material may be millimolar or higher. Binding affinity can be in the micromolar range (kD=10⁻⁴ to 10⁻⁶), nanomole range (kD=10⁻⁷ to 10⁻⁹), picomole range (kD=10⁻¹⁰ to 10⁻¹²), or femtomole range (kD=10⁻¹³ to 10⁻¹⁵).

The affinity of an antibody for its target (e.g., antigen or epitope) can be at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target can be characterized by a K_{d} of, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution.

As used herein, an antibody “binds” or “recognizes” an antigen or epitope if it binds the antigen or epitope with a Kd of less than 10⁻⁴ M. The term “binds” with respect to a cell type (e.g., an antibody that binds cancer cells), typically indicates that an agent binds a majority of the cells in a pure population of those cells. For example, an antibody that binds a given cell type typically binds to at least ⅔ of the cells in a population of the indicated cells (e.g., 67, 75, 80, 85, 90, 91, 92, 93, 94, 95, 96, 97, 98, 99, or 100%) if the antibody is in high enough concentration. In some cases, binding to a polypeptide can be assayed by comparing binding of the antibody to a cell that presents the polypeptide to binding (or lack thereof) of the antibody to a cell that does not express the polypeptide. One of skill will recognize that some variability will arise depending on the method and/or threshold of determining binding. Affinity of an antibody for a target can be determined according to methods known in the art, e.g., as reviewed in Ernst et al. Determination of Equilibrium Dissociation Constants, Therapeutic Monoclonal Antibodies (Wiley & Sons ed. 2009).

As used herein, an antibody “specifically binds” or is “specific for” an antigen or epitope if it binds the antigen or epitope with at least 2-fold greater affinity it does non-antigens or non-epitopes, e.g., with at least any of 4-fold, 5-fold, 6-fold, 7-fold, 8-fold, 9-fold, 10-fold, 20-fold, 25-fold, 50-fold, or 100-fold greater affinity. For example, a monoclonal antibody raised against a first peptide epitope may specifically bind the first peptide epitope but may not specifically bind a second, different peptide epitope, even if the amino acid sequence of the two epitopes partially overlap. In this situation, the monoclonal antibody may exhibit some binding affinity for the second peptide epitope. However, this affinity typically is less than half the affinity for the first peptide epitope.

The term “captures” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 67, 75, 80, 85, 90, 91, 92, 93, 94, 95, 96, 97, 98, 99, or 100%). One of skill will recognize that some variability will arise depending on the method and/or threshold of determining binding.

As used herein, the term “monoclonal antibody” refers to a clonal preparation or composition of antibodies with a single binding specificity and affinity for a given epitope on an antigen (“monoclonal antibody composition”). A “polyclonal antibody” refers to a preparation or composition of antibodies that are raised against a single antigen, but with different binding specificities and affinities (“polyclonal antibody composition”).

As used herein, the term “chimeric antibody” refers to an antibody in which (a) the constant region, or a portion thereof, is altered, replaced or exchanged so that the antigen binding site (variable region, CDR, or portion thereof) is linked to a constant region of a different or altered class, effector function and/or species; or (b) the variable region, or a portion thereof, is altered, replaced or exchanged with a variable region having a different or altered antigen specificity (e.g., CDR and framework regions from different species). Chimeric antibodies can include variable region fragments, e.g., a recombinant antibody comprising two Fab or Fv regions or an scFv. A chimeric antibody can also, as indicated above, include an Fc region from a different source than the attached Fv regions. In some cases, the chimeric antibody includes chimerism within the Fv region. An example of such a chimeric antibody would be a humanized antibody where the Fvs and CDRs are from different sources.

As used herein, the term “humanized antibody” refers to an antibody in which the antigen binding loops, i.e., CDRs, obtained from the VH and VL regions of a non-human antibody are grafted to a human framework sequence. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996). Transgenic mice, or other organisms such as other mammals, may also be used to express humanized or human antibodies, as disclosed in U.S. Pat. No. 6,673,986.

As used herein, an antibody is “monospecific” if all of its antigen binding sites bind to the same epitope.

As used herein, an antibody is “bispecific” if it has at least two different antigen binding sites which each bind to a different epitope.

As used herein, the term “conjugate” refers to a first molecule, e.g., an antibody, chemically coupled with a moiety, such as a detectable label or a biologically active moiety, such as a drug, toxin or chemotherapeutic agent. Accordingly, this disclosure contemplates antibodies conjugated with one or more moieties.

As used herein, the term “labeled” molecule (e.g., nucleic acid, protein, or antibody) refers to a molecule that is bound to a detectable label, either covalently, through a linker or a chemical bond, or noncovalently, through ionic, van der Waals, electrostatic, or hydrogen bonds, such that the presence of the molecule may be detected by detecting the presence of the detectable label bound to the molecule.

As used herein, the term “detectable label” refers to a composition detectable by spectroscopic, photochemical, biochemical, immunochemical, chemical, or other physical means. Examples of detectable labels are described herein and include, without limitation, colorimetric, fluorescent, chemiluminescent, enzymatic, and radioactive labels. For the purposes of the present disclosure, a detectable label can also be a moiety that does not itself produce a signal (e.g., biotin), but that binds to a second moiety that is able to produce a signal (e.g., labeled avidin).

The term “cross-linked” with respect to an antibody refers to attachment of the antibody to a solid or semisolid matrix (e.g., sepharose, beads, microtiter plate), or to another protein or antibody. For example, an antibody can be multimerized to create an antibody complex with multiple (more than 2) antigen-binding sites. The antibody can be multimerized by expressing the antibody as a high-valency isotype (e.g., IgA or IgM, which typically form complexes of 2 or 5 antibodies, respectively). Antibody multimerization can also be carried out by using a cross-linker comprising a reactive group capable of linking proteins (e.g., carbodiimide, NHS esters, etc.). Methods and compositions for cross-linking an antibody to a matrix are described, e.g., in the Abcam and New England Biolab catalogs and websites (available at abcam.com and neb.com). Cross-linker compounds with various reactive groups are described, e.g., in Thermo Fisher Scientific catalog and website (available at piercenet.com).

As used herein, the term “immunoassay” refers to a method for detecting an analyte by detecting binding between an antibody that recognizes the analyte and the analyte.

As used herein, the term “antibody-drug conjugate” or (“ADC”) refers to an antibody conjugated with a drug. Typically, conjugation involves covalent binding through a linker.

As used herein, the term “expression construct” refers to a polynucleotide comprising an expression control sequence operatively linked with a nucleotide sequence that is to be the subject of expression. As used herein, the term “expression vector” refers to a polynucleotide comprising an expression construct and sequences sufficient for replication in a host cell or insertion into a host chromosome. Plasmids and viruses are examples of expression vectors. As used herein, the term “expression control sequence” refers to a nucleotide sequence that regulates transcription and/or translation of a nucleotide sequence operatively linked thereto. Expression control sequences include promoters, enhancers, repressors (transcription regulatory sequences) and ribosome binding sites (translation regulatory sequences).

As used herein, a nucleotide sequence is “operatively linked” with an expression control sequence when the expression control sequence functions in a cell to regulate transcription of the nucleotide sequence. This includes promoting transcription of the nucleotide sequence through an interaction between a polymerase and a promoter.

As used herein, the term “biological sample” refers to a sample containing cells or biological molecules derived from cells. A biological sample can be obtained from a subject, e.g., a patient, from an animal, such as an animal model, or from cultured cells, e.g., a cell line or cells removed from a patient and grown in culture for observation. A biological sample can comprise tissue and/or liquid. It can be obtained from any biological source including without limitation blood, a blood fraction (e.g., serum or plasma), cerebrospinal fluid (CSF), lymph, saliva, sputum, buccal swab, urine or feces. A biological sample can be a biopsy, such as a tissue biopsy, such as needle biopsy, fine needle biopsy, surgical biopsy, etc. The sample can comprise a tissue sample harboring a lesion or suspected lesion, although the biological sample may also be derived from another site, e.g., a site of suspected metastasis, a lymph node, or from the blood. A biological sample can be a fraction of a sample taken from a subject.

As used herein, the term “diagnosis” refers to a relative probability that a subject has a disorder such as cancer. Similarly, the term “prognosis” refers to a relative probability that a certain future outcome may occur in the subject. For example, in the context of the present disclosure, prognosis can refer to the likelihood that an individual will develop cancer, have recurrence, that the cancer will metastasize, that the cancer will be cured, or the likely severity of the disease (e.g., severity of symptoms, rate of functional decline, survival, etc.). The terms are not intended to be absolute, as will be appreciated by any one of skill in the field of medical diagnostics.

As used herein, the term terms “therapy,” “treatment,” “therapeutic intervention” and “amelioration” refer to any activity resulting in a reduction in the severity of symptoms. In the case of cancer, treatment can refer to, e.g., reducing tumor size, number of cancer cells, growth rate, metastatic activity, reducing cell death of non-cancer cells, reduced nausea and other chemotherapy or radiotherapy side effects, etc. The terms “treat” and “prevent” are not intended to be absolute terms. Treatment and prevention can refer to any delay in onset, amelioration of symptoms, improvement in patient survival, increase in survival time or rate, etc. Treatment and prevention can be complete (undetectable levels of neoplastic cells) or partial, such that fewer neoplastic cells are found in a patient than would have occurred without the present intervention. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

As used herein, the terms “inhibits” or “inhibition” (e.g., inhibition of characteristics such as cellular growth, proliferation, metabolic activity, viability, survival or division) refer to a decrease in the characteristic relative to a control. In some cases, a compound of the present invention may inhibit one of the foregoing characteristics. For example, a cell treated with an antibody, peptide epitope or polytope described herein may exhibit a decrease in one of the foregoing characteristics of at least 5%, 10%, 25%, 50%, 80%, 90%, 95%, 97.5%, 99%, 99.5%, 99.9% as compared to an untreated cell. In some cases, a cell treated with a compound, e.g., an antibody-drug conjugate, of the present invention may exhibit a growth, proliferation, metabolic activity, viability, survival or division that is less than about 5%, 10%, 25%, 50%, 80%, 90%, 95%, 97.5%, 99%, 99.5%, 99.9% of a control (e.g. untreated) cell.

As used herein, the term “pharmaceutical composition” refers to a composition comprising a pharmaceutical compound (e.g., a drug) and a pharmaceutically acceptable carrier.

As used herein, the term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte A™ (Baxter).

Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable label (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

As used herein, the term “subject” refers to an individual animal. The term “patient” as used herein refers to a subject under the care or supervision of a health care provider such as a doctor or nurse. Subjects include mammals, such as humans and non-human primates, such as monkeys, as well as dogs, cats, horses, bovines, rabbits, rats, mice, goats, pigs, and other mammalian species. Subjects can also include avians. A patient can be an individual that is seeking treatment, monitoring, adjustment or modification of an existing therapeutic regimen, etc. The term “cancer subject” refers to an individual that has been diagnosed with cancer. Cancer patients can include individuals that have not received treatment, are currently receiving treatment, have had surgery, and those that have discontinued treatment.

In the context of treating cancer, a subject in need of treatment can refer to an individual that has cancer or a pre-cancerous condition, has had cancer and is at risk of recurrence, is suspected of having cancer, is undergoing standard treatment for cancer, such as radiotherapy or chemotherapy, etc.

“Cancer”, “tumor,” “transformed” and like terms include precancerous, neoplastic, transformed, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3^{rd} ed. 1994)). Although transformation can arise from infection with a transforming virus and incorporation of new genomic DNA, or uptake of exogenous DNA, it can also arise spontaneously or following exposure to a carcinogen.

The term “cancer” can refer to any cancer, including those in Table 1. This includes, without limitation, leukemias, carcinomas, sarcomas, adenocarcinomas, lymphomas, solid and lymphoid cancers, etc. Examples of different types of cancer include, but are not limited to, lung cancer (e.g., non-small cell lung cancer or NSCLC), breast cancer, prostate cancer, colorectal cancer, bladder cancer, ovarian cancer, leukemia, liver cancer (i.e., hepatocarcinoma), renal cancer (i.e., renal cell carcinoma), thyroid cancer, pancreatic cancer, uterine cancer, cervical cancer, testicular cancer, esophageal cancer, stomach (gastric) cancer, kidney cancer, cancer of the central nervous system, skin cancer, glioblastoma and melanoma.

TABLE 1
Cancers

Bone and muscle
Chondrosarcoma
Ewing's sarcoma
Malignant fibrous histiocytoma of bone/osteosarcoma
Osteosarcoma
Rhabdomyosarcoma
Heart cancer
Brain and nervous system
Astrocytoma
Brainstem glioma
Pilocytic astrocytoma
Ependymoma
Primitive neuroectodermal tumor
Cerebellar astrocytoma
Cerebral astrocytoma
Glioma
Medulloblastoma
Neuroblastoma
Oligodendroglioma
Pineal astrocytoma
Pituitary adenoma
Visual pathway and hypothalamic glioma
Breast
Breast cancer
Invasive lobular carcinoma
Tubular carcinoma
Invasive cribriform carcinoma
Medullary carcinoma
Male breast cancer
Phyllodes tumor
Inflammatory Breast Cancer
Endocrine system
Adrenocortical carcinoma
Islet cell carcinoma (endocrine pancreas)
Multiple endocrine neoplasia syndrome
Parathyroid cancer
Pheochromocytoma
Thyroid cancer
Merkel cell carcinoma
Eye
Uveal melanoma
Retinoblastoma
Gastrointestinal
Anal cancer
Appendix cancer
cholangiocarcinoma
Carcinoid tumor, gastrointestinal
Colon cancer
Extrahepatic bile duct cancer
Gallbladder cancer
Gastric (stomach) cancer
Gastrointestinal carcinoid tumor
Gastrointestinal stromal tumor (GIST)
Hepatocellular cancer
Pancreatic cancer, islet cell
Rectal cancer
Genitourinary and gynecologic
Bladder cancer
Cervical cancer
Endometrial cancer
Extragonadal germ cell tumor
Ovarian cancer
Ovarian epithelial cancer (surface epithelial-stromal tumor)
Ovarian germ cell tumor
Penile cancer
Renal cell carcinoma
Renal pelvis and ureter, transitional cell cancer
Prostate cancer
Testicular cancer
Gestational trophoblastic tumor
Ureter and renal pelvis, transitional cell cancer
Urethral cancer
Uterine sarcoma
Vaginal cancer
Vulvar cancer
Wilms tumor
Head and neck
Esophageal cancer
Head and neck cancer
Nasopharyngeal carcinoma
Oral cancer
Oropharyngeal cancer
Paranasal sinus and nasal cavity cancer
Pharyngeal cancer
Salivary gland cancer
Hypopharyngeal cancer
Hematopoetic
Acute biphenotypic leukemia
Acute eosinophilic leukemia
Acute lymphoblastic leukemia
Acute myeloid leukemia
Acute myeloid dendritic cell leukemia
AIDS-related lymphoma
Anaplastic large cell lymphoma
Angioimmunoblastic T-cell lymphoma
B-cell prolymphocytic leukemia
Burkitt's lymphoma
Chronic lymphocytic leukemia
Chronic myelogenous leukemia
Cutaneous T-cell lymphoma
Diffuse large B-cell lymphoma
Follicular lymphoma
Hairy cell leukemia
Hepatosplenic T-cell lymphoma
Hodgkin's lymphoma
Hairy cell leukemia
Intravascular large B-cell lymphoma
Large granular lymphocytic leukemia
Lymphoplasmacytic lymphoma
Lymphomatoid granulomatosis
Mantle cell lymphoma
Marginal zone B-cell lymphoma
Mast cell leukemia
Mediastinal large B cell lymphoma
Multiple myeloma/plasma cell neoplasm
Myelodysplastic syndromes
Mucosa-associated lymphoid tissue lymphoma
Mycosis fungoides
Nodal marginal zone B cell lymphoma
Non-Hodgkin lymphoma
Precursor B lymphoblastic leukemia
Primary central nervous system lymphoma
Primary cutaneous follicular lymphoma
Primary cutaneous immunocytoma
Primary effusion lymphoma
Plasmablastic lymphoma
Sézary syndrome
Splenic marginal zone lymphoma
T-cell prolymphocytic leukemia
Skin
Basal-cell carcinoma
Melanoma
Skin cancer (non-melanoma)
Thoracic and respiratory
Bronchial adenomas/carcinoids
Small cell lung cancer
Mesothelioma
Non-small cell lung cancer
Pleuropulmonary blastoma
Laryngeal cancer
Thymoma and thymic carcinoma
HIV/AIDS related
AIDS-related cancers
Kaposi sarcoma
Unsorted
Epithelioid hemangioendothelioma (EHE)
Desmoplastic small round cell tumor
Liposarcoma

With respect to diagnosis of cancer, it is important to note that cancer is not a single disease, but is rather a heterogeneous collection of diseases; and that such heterogeneity extends to the spectrum of genes expressed in each individual case of any particular cancer. See, for example, Alberts et al., “Molecular Biology of the Cell,” Fifth Edition, Garland Science, 2008, pages 1256 and 1264. Due to this known heterogeneity that is characteristic of all cancers, different individuals suffering from a particular cancer will have different sets of genes that are overexpressed and/or underexpressed. Accordingly, there is no natural law that specifies the particular set of genes that is expressed in all instances of a particular cancer.

The terms “sensitivity”, “specificity” and “accuracy” as used herein refer to measures of agreement. Sensitivity is the percentage of actual positives identified in a test as positive. Sensitivity includes, for example, instances in which one should have found cancer in a subject, e.g., by assaying AABH) and did (e.g., as verified by sampling cellular DNA or tumor tissue). (Sensitivity=TP/(TP+FN.) Specificity is the percentage of actual negatives identified in a test as negative. Specificity includes, for example, instances in which one should have found no cancer in a subject (e.g., as predicted by assaying AABH) and did not (e.g., as verified by sampling cellular DNA or tumor tissue). Specificity can be calculated using the following equation: Specificity=TN/(TN+FP). Subjects identified as positive in a test that are in reality positive are referred as true positives (TP). Subjects identified as positive in a test that are in reality negative are referred as false positives (FP). Subjects identified as negative in a test that are in reality negative are referred as true negatives (TN). Subjects identified as negative in a test that are in reality positive are referred as false negatives (FN). Accuracy can be measured by the percentage of tests taken that give a correct result. Accuracy=(TP+TN)/(TP+FP+TN+FN).

Positive predictive value (PPV) can be measured by the percentage of subjects who test positive that are true positives. PPV can be calculated using the following equation: PPV=TP/(TP+FP), where TP are true positives and FP are false positives.

### II. Polypeptide Epitopes of Aspartyl (Asparaginyl) β-Hydroxylase

Provided herein are polypeptide epitopes presented by HLA proteins on the surface of cells expressing AABH. These polypeptides have amino acid sequences consisting of or consisting essentially of, or that are functional equivalents of, the sequences:

SEQ ID NO: 1
CRRGQIKYS;
SEQ ID NO: 2
GPTNCRLRMHLGLVI;
SEQ ID NO: 3
RTWEEGKVLIFD;
SEQ ID NO: 4
WQDASSFRLIFI;
SEQ ID NO: 5
LSGTSFFTWFMVIALLGVWTSV;
SEQ ID NO: 6
VYEEVLSVTPNDGFAKVHYGFILKAQNK;
SEQ ID NO: 7
FASVWQRSLYNVNGLK;
SEQ ID NO: 8
GLSGTSFFTWFMVIALLGVWTSVA;
SEQ ID NO: 9
GDGDFDVDDAKVLLGLKERSTSEPAV;
SEQ ID NO: 10
IEEAVNAFKELVRKYPQSPRARYGKAQC;
SEQ ID NO: 11
DVPADLLKLSLKRRSDRQQFLGH;
SEQ ID NO: 12
SLERNWKLIRDEGLAVMDKAKGL;
SEQ ID NO: 13
GFAKVHYGFILKAQNKIAESIP;
SEQ ID NO: 14
HTGPTNCRLRMHLGLVIPKEGC;
SEQ ID NO: 15
DSFEHEVWQDASSFRLIFIVDVW;
SEQ ID NO: 16
MRGSLLTLQRLVQLFPNDTSLKN;
SEQ ID NO: 17
PQQEDDEFLMATDVDDRFETL;
SEQ ID NO: 18
DGRFYFHLGDAMQRVGNKEAY;
SEQ ID NO: 19
GLSGTSFFTWFMVIALLGVWTSVAGGSGGGFAKVHYGFILKAQNKIAESI
P;
SEQ ID NO: 20
KVYEEVLSVE EVLSVTPNTP NDGFAKVGFA KVHYGFKVHY
GFILKKIAES IPYL;
SEQ ID NO: 21
GTDDGRFYFRVGNKEAYKASVWQRSLYSLYNVNGLK;
SEQ ID NO: 22
SFFTWFMVIALLGVWTSVA;
SEQ ID NO: 23
PADLLKLSLKRRSDRQQF;
SEQ ID NO: 24
GFAKVHYGFILKAQNKIAESIPY;
SEQ ID NO: 25
TGPTNCRLRMHLGLVIPKEGC;
SEQ ID NO: 26
FEHEVWQDASSFRLIFVDVWHPEL;
SEQ ID NO: 27
EHVEGEDLQQEDGPTGEPQQEDDEFL;
SEQ ID NO: 28
PYLKEGIESGDPGTDDGR;
SEQ ID NO: 29
GLKAQPWWTPKETGYTE;
SEQ ID NO: 30
LKAQNKIAESIPYLKEGI;
SEQ ID NO: 31
HLGDAMQRVGNKEAYKWYELGHKRGHFASVW;
SEQ ID NO: 32
VDVWHPELTPQQRRSLPAI;
SEQ ID NO: 33
KNAKSSGNSSSSGSGSGSTSAGSSSPGARRE;
SEQ ID NO: 34
IYDADGDGDFDVDDAKVLLGLKERSTSEPAVP;
SEQ ID NO: 35
EEMMSEQENPDSSEPVVE
and
SEQ ID NO: 36
FPVEEQQEVPPETNRKTDDPEQKAKVKKKK.

These polypeptides are identified using a neural network training method to predict T-cell epitopes of the AABH amino acid sequence of SEQ ID NO: X for different human alleles of human major histocompatibility complex I (MHC I) receptors. This includes, for example, HLA-a*01:01, HLA-a*02:01, HLA A*02:06, etc. These polypeptide epitopes are useful for eliciting antibodies that bind AABH.

In certain embodiments, animals are immunized with AABH Fragment 1 or AABH Fragment 2, as follows:

AABH Fragment 1:
(SEQ ID NO: 38)
LDAAEK LRKRGKIEEA VNAFKELVRK YPQSPRARYG KAQCEDDLAE
KRRSNEVLRG AIETYQEVAS LPDVPADLLK LSLKRRSDRQ
QFLGHMRGSL LTLQRLVQLF PNDTSLKNDL GVGYLLIGDN
DNAKKVYEEV LSVTPNDGFA KVHYGFILKA QNKIAESIPY
LKEGIESGDP GTDDGRFYFH LGDAMQRVGN KEAYKWYELG
HKRGHFASVW Q RSLYNVNGL KAQPWWTPKE TGYTELVKSL
ERNWKLIRDE GLAVMDKAKG LFLPEDENLR EKGDWSQFTL
WQQGRRNENA CKGAPKTCTL LEKFPETTGC RRGQIKYSIM
HPGTHVWPHT GPTNCRLRMH LGLVIPKEGC KIRCANETRT
WEEGKVLIFD DSFEHEVWQD ASSFRLIFIV DVWHPELTPQ
QRRSLPAI;
and
AABH Fragment 2:
(SEQ ID NO: 39)
ASSFRLIFIV DVWHPELTPQ QRRSLPAI.

In certain embodiments polypeptide epitopes of this disclosure can be provided in substantially pure form. This can include, for example, a solution comprising a solvent, a buffer and the polypeptide epitope, e.g., a pharmaceutical composition. It also can include a composition comprising polypeptide epitope and a pharmaceutically acceptable carrier, for example, an adjuvant.

In addition to binding the human cognate, the antibodies of this disclosure may bind to aspartyl (asparaginyl) β-hydroxylase of any mammal, including, without limitation, chimp, primate, artiodactyla, carnivora, cetacean, chiroptera, dermoptera, edentata, hyracoidae, hyracoidae, insectivore, lagomorpha, marsupialia, monotremata, perissodactyla, ungulate, pholidata and pinnipedia.

Polypeptides and peptide epitopes of this disclosure can be produced using any methods known in the art to synthesize polypeptides, including recombinant and non-recombinant methods (e.g., chemical synthesis). Chemical synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject polypeptide. Various forms of SPPS, such as Fmoc and t-Boc, are available. Techniques for solid-phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963); Stewart et al., Solid Phase Peptide Synthesis, 2nd ed. Pierce Chem. Co., Rockford, III. (1984); Ganesan A. 2006 Mini Rev. Med Chem. 6:3-10; Camarero J A et al. 2005 Protein Pept Lett. 12:723-8; and Methods in Molecular Biology, Vol 35: Peptide Synthesis Protocols. Briefly, small insoluble, porous beads are treated with functional units on which peptide chains are built. After repeated cycling of coupling/deprotection, the free N-terminal amine of a solid-phase attached nascent peptide is coupled to a single N-protected amino acid unit. This unit is then deprotected, revealing a new N-terminal amine to which a further amino acid may be attached. The peptide remains immobilized on the solid-phase and undergoes a filtration process before being cleaved from the support.

The present disclosure further contemplates recombinant host cells containing an exogenous polynucleotide encoding one or more of the peptides or polypeptides disclosed herein. For peptides and polypeptides produced using recombinant techniques, the methods can involve any suitable construct and any suitable host cell, which can be a prokaryotic or eukaryotic cell (e.g., a bacterial or yeast host cell). Methods for introducing genetic material into host cells include, for example, transformation, electroporation, conjugation, calcium phosphate precipitation methods and the like. The method for transfer can be selected so as to provide for stable expression of the introduced polypeptide-encoding nucleic acid. The polypeptide-encoding nucleic acid can be provided as a heritable episomal element (e.g., plasmid) or can be genomically integrated.

Suitable vectors for incorporating polypeptide-encoding nucleic acids can vary in composition. Integrative vectors can be conditionally replicative or suicide plasmids, bacteriophages, and the like. The constructs can include various elements, including for example, promoters, selectable genetic markers (e.g., genes conferring resistance to antibiotics (for instance kanamycin, erythromycin, chloramphenicol, or gentamycin)), origin of replication (to promote replication in a host cell, e.g., a bacterial host cell), and the like. The choice of vector will depend upon a variety of factors such as the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal. The choice of appropriate vector is well within the skill of the art. Many such vectors are available commercially.

The vector used may be an expression vector based on an episomal plasmid containing one or more selectable drug resistance markers and elements that provide for autonomous replication in different host cells. Vectors are amply described in numerous publications well known to those in the art, including, e.g., Short Protocols in Molecular Biology, (1999) F. Ausubel, et al., eds., Wiley & Sons. Vectors may provide for expression of the nucleic acids encoding the subject polypeptide, may provide for propagating the subject nucleic acids, or both.

Constructs can be prepared by, for example, inserting a polynucleotide of interest into a construct backbone, typically by means of DNA ligase attachment to a cleaved restriction enzyme site in the vector. Alternatively, the desired nucleotide sequence can be inserted by homologous recombination or site-specific recombination or by amplification methods such as the polymerase chain reaction (PCR). Typically homologous recombination is accomplished by attaching regions of homology to the vector on the flanks of the desired nucleotide sequence, while site-specific recombination can be accomplished through use of sequences that facilitate site-specific recombination (e.g., cre-lox, att sites, etc.). Nucleic acids containing such sequences can be added by, for example, ligation of oligonucleotides, or by polymerase chain reaction using primers comprising both the region of homology and a portion of the desired nucleotide sequence.

For expression of a polypeptide of interest, an expression cassette may be employed. Thus, the present disclosure provides a recombinant expression vector comprising a nucleic acid encoding a polypeptide; e.g., a peptide epitope. The expression vector provides transcriptional and translational regulatory sequences, and may provide for inducible or constitutive expression, where the coding region is operably linked under the transcriptional control of the transcriptional initiation region and a transcriptional termination region. These control regions can be derived from exogenous sources, such as viruses or cellular genes. As such, control regions from exogenous sources may be considered heterologous elements that are operably linked to the nucleic acid encoding the subject polypeptide. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7, and the like).

Expression vectors generally have convenient restriction sites located near the promoter sequence to provide for the insertion of nucleic acid sequences encoding proteins of interest. A selectable marker operative in the expression host may be present to facilitate selection of cells containing the vector. In addition, the expression construct may include additional elements. For example, the expression vector may have one or two replication systems, thus allowing it to be maintained in organisms, for example in mammalian or insect cells for expression and in a prokaryotic host for cloning and amplification. In addition, the expression construct may contain a selectable marker gene to allow the selection of transformed host cells. Selection genes are well known in the art and will vary with the host cell used.

It should be noted that polypeptide of the present disclosure may be synthesized to contain additional elements, such as a detectable label, e.g., a radioactive label, a fluorescent label, a biotin label, an immunologically detectable label (e.g., a hemagglutinin (HA) tag, a poly-Histidine tag), a nuclear localization sequence, and the like. Additional elements of the subject polypeptide can be provided to facilitate expression (e.g., N-terminal methionine and/or heterologous signal sequence to facilitate expression in host cells), isolation (e.g., biotin tag, immunologically detectable tag; e.g., a myc tag or a His₆ tag) through various methods (e.g., affinity capture, etc.). The polypeptide may also optionally be immobilized on a support through covalent or non-covalent attachment.

Isolation and purification of the subject peptide or polypeptide is accomplished according to methods known in the art. For example, the peptide or polypeptide can be isolated from a lysate of genetically modified cells, from supernatant of cell culture media, or from a synthetic reaction mix, by immunoaffinity purification, which generally involves contacting the sample with an antibody against an epitope of the polypeptide, washing to remove non-specifically bound material, and eluting specifically bound polypeptide. Isolated polypeptide can be further purified by dialysis and other methods normally employed in protein purification, e.g. ion-exchange, sizing and/or metal chelate chromatography methods.

### III. Chimeric Polyepitopes

Also provided herein are chimeric polyepitopes of AABH. Polyepitopes provided herein are polypeptides having an amino acid sequences comprising a plurality of AABH polypeptide epitopes linked through peptide or chemical linkers. The polypeptide epitopes can be selected from any two, three, four, or five polypeptide epitopes as described herein. The peptide linkers can comprise at least four and no more than 12 amino acids, e.g., no more than or exactly 5, 6, 7, 8, 9, 10, 11 or 12 amino acids. In one embodiment, the peptide linker has the amino acid sequence GGSGG. Additional linkers include, but are not limited to, polyethylene glycol (PEG), β-alanine, 4-aminobutyric acid (GABA), (2-aminoethoxy)acetic acid (AEA), 5-aminovaleric acid (AVA), 6-aminocaproic acid (Ahx), 8-amino-3,6-dioxaoctanoic acid (mini-PEG1), 12-amino-4,7,10-trioxadodecanoic acid (mini-PEG2), 15-amino-4,7,10,13-tetraoxapentadecanoic acid (mini-PEG3) and trioxatridecansuccinamic acid. See also Chen et al. (2013) Adv. Drug Deliv. Rev. 65:1357-1369.

In certain embodiments, polyepitopes (or “polytopes”) have any of the following amino acid sequences:

(SEQ ID NO: 40)
GLSGTSFFTWFMVIALLGVWTSVAGGSGGGFAKVHYGFILKAQNKIAESI
PGGSGGHTGPTNCRLRMHLGLVIPKEGC;
(SEQ ID NO: 41)
GLSGTSFFTWFMVIALLGVWTSVAGGSGGIEEAVNAFKELVRKYPQSPRA
RYGKAQCGGSGGDVPADLLKLSLKRRSDRQQFLGH;
(SEQ ID NO: 42)
DVPADLLKLSLKRRSDRQQFLGHGGSGGSLERNWKLIRDEGLAVMDKAKG
L;
(SEQ ID NO: 43)
GLSGTSFFTWFMVIALLGVWTSVAGGSGGGFAKVHYGFILKAQNKIAESI
P.

Polyepitopes also can be provided in substantially pure form and in pharmaceutically acceptable carriers comprising, e.g., adjuvants.

### IV. Hybridomas

Also provided herein are hybridomas that produce monoclonal antibodies of this disclosure, and methods of making. In general, production of hybridomas involves: a) immunizing animals with the target immunogen; b) isolating B cells from the animals; c) fusing the B cells with myeloma cells to produce hybridomas and d) selecting hybridomas that produce monoclonal antibodies against the immunogen.

Laboratory animals such as mice, rats, goats or sheep are immunized with an immunogen of interest. In the present case, the immunogen can be a peptide epitope, chimeric immunogen or polypeptide as disclosed herein. The animal can be subject to a plurality of inoculations given over a period of time, such as several weeks. In vivo electroporation can facilitate the immune response.

After development of an immune response, animals are sacrificed and their spleens removed. B cells are isolated from the spleen.

Isolated B cells are fused with myeloma cells to produce hybridomas. Typically, the myeloma cells used are sensitive to a toxin, such as hypoxanthine-aminopterin (HAT). For example, such cells can lack the hypoxanthine-guanine phosphoribosyltransferase (HGPRT) gene. Hybridomas are cultured in a medium containing the toxin, such as HAT, to kill unfused myeloma cells. Incubation can be for a time sufficient to kill unfused myeloma cells and unfused B cells, e.g., about ten to fourteen days.

The surviving hybridoma cells are screened for production of the monoclonal antibodies of interest. Screening can involve plating hybridoma cells into a multiwell plate so that each well contains one hybridoma. After production and secretion of antibodies into the supernatant, the supernatant is screened by ELISA or other immunoassay to determine whether the monoclonal antibodies bind to the immunogen.

The selected hybridoma cells are then cloned. Cloning can involve culturing cells with interleukin-6. The cells can be maintained in in culture medium such a RPMI-1640 to produce antibodies.

### V. Monoclonal Antibodies

Also provided herein are monoclonal antibodies that specifically bind to a polypeptide epitope or a chimeric polyepitope of this disclosure. In certain embodiments, a monoclonal antibody of this disclosure binds a polypeptide epitope described herein with greater affinity than either (1) a polypeptide comprising the same epitope embedded in a longer amino acid sequence or (2) a polypeptide comprising a portion of the same epitope, i.e., that overlaps with the amino acid sequences of the polypeptide epitopes disclosed herein. Similarly, monoclonal antibodies raised against polypeptides having amino acid sequences that do not consist of the amino acid sequences of the polypeptide epitopes disclosed herein bind the polypeptide epitopes disclosed herein with lower affinity than they do the antigens against which they were raised. Monoclonal antibodies specific for the polypeptide epitopes and chimeric polyepitopes described herein also bind AABH protein, e.g., with affinity at least in the micromolar or nanomolar ranges.

For preparation of the presently described antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 0308936).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus. Antibodies also can be produced by culturing hybridomas, as described herein.

An antibody of the invention can also be produced in various formats, including as a Fab, a Fab′, a F(ab′)₂, a scFv, Fd or a sdAb. The antibody fragments can be obtained by a variety of methods, including, digestion of an intact antibody with an enzyme, such as pepsin (to generate (Fab′)₂ fragments) or papain (to generate Fab fragments); or by de novo peptide synthesis. Antibody fragments can also be synthesized using recombinant DNA methodology. An antibody of the invention can also include a human constant region. See, e.g., Fundamental Immunology (Paul ed., 4d ed. 1999); Bird, et al., Science 242:423 (1988); and Huston, et al., Proc. Natl. Acad. Sci. USA 85:5879 (1988).

Methods for humanizing non-human antibodies (i.e., using CDRs from non-human antibodies) are also known in the art. Generally, a humanized antibody has one or more amino acid residues from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In some cases, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007). The antibody or antibody fragment can also be labeled, or conjugated to a therapeutic agent as described herein.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

In some embodiments, a subject antibody comprises one or more non-naturally occurring amino acids. In some embodiments, the non-naturally occurring amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for exemplary non-naturally occurring amino acids. Inclusion of a non-naturally occurring amino acid can provide for linkage to a polymer, a second polypeptide, a scaffold, etc. For example, a subject antibody linked to a water-soluble polymer can be made by reacting a water-soluble polymer (e.g., PEG) that comprises a carbonyl group to an the subject antibody that comprises a non-naturally occurring amino acid that comprises an aminooxy, hydrazine, hydrazide or semicarbazide group. As another example, a subject antibody linked to a water-soluble polymer can be made by reacting a subject antibody that comprises an alkyne-containing amino acid with a water-soluble polymer (e.g., PEG) that comprises an azide moiety; in some embodiments, the azide or alkyne group is linked to the PEG molecule through an amide linkage.

A “non-naturally occurring amino acid” refers to an amino acid that is not one of the 20 common amino acids found in naturally-occurring proteins, or pyrolysine or selenocysteine. Other terms that may be used synonymously with the term “non-naturally occurring amino acid” are “non-natural amino acid,” “unnatural amino acid,” “non-naturally-encoded amino acid,” and variously hyphenated and non-hyphenated versions thereof. The term “non-naturally occurring amino acid” also includes, but is not limited to, amino acids that occur by modification (e.g., post-translational modifications) of a naturally occurring amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

In some embodiments, a subject antibody is linked (e.g., covalently linked) to a non-peptide polymer (e.g., a polymer other than a polypeptide). Suitable polymers include, e.g., biocompatible polymers, and water-soluble biocompatible polymers, synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g. a homo- or hetero-polysaccharide. Additional polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, acetonitrile butadiene styrene (ABS) resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50,000 Da, e.g., from 5,000 Da to 40,000 Da, or from 25,000 to 40,000 Da. For example, in some embodiments, in which a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

As noted above, in some embodiments, a subject antibody is covalently linked to a PEG polymer. See, e.g., Albrecht et al. (2006) J. Immunol. Methods 310:100. Methods and reagents suitable for PEGylation of a protein are well known in the art and may be found in, e.g., U.S. Pat. No. 5,849,860. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbon atoms.

The PEG conjugated to the subject antibody can be linear or branched. Branched PEG derivatives are described, for example, in U.S. Pat. No. 5,643,575, “star-PEG'S” and multi-armed PEG'S are described, for example, in Shearwater Polymers, Inc. catalog “Polyethylene Glycol Derivatives 1997-1998.” Star PEGs are described in the art including, e.g., in U.S. Pat. No. 6,046,305.

A subject antibody can be glycosylated, e.g., can comprise a covalently linked carbohydrate or polysaccharide moiety. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to an antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of an antibody.

A subject antibody will in some embodiments comprise a “radiopaque” label, e.g., a label that can be easily visualized using for example X-rays. Radiopaque materials are well known to those of skill in the art. The most common radiopaque materials include iodide, bromide or barium salts. Other radiopaque materials are also known and include, but are not limited to, organic bismuth derivatives (see, e.g., U.S. Pat. No. 5,939,045), radiopaque multiurethanes (see U.S. Pat. No. 5,346,981), organobismuth composites (see, e.g., U.S. Pat. No. 5,256,334), radiopaque barium multimer complexes (see, e.g., U.S. Pat. No. 4,866,132), and the like.

A subject antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional N-hydroxysuccinimide (NHS) esters and imido esters cross-link amine containing polypeptides. At mildly alkaline pH, imido esters react only with primary amines to form imidoamides, and the overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A subject antibody can be immobilized on (i.e., affixed to) a solid support. Suitable supports are well known in the art and comprise, inter alia, commercially available column materials, particles, polystyrene beads, latex beads, magnetic beads, colloidal metal particles, glass and/or silicon chips and surfaces, nitrocellulose strips, nylon membranes, sheets, duracytes, wells of reaction trays (e.g., multi-well plates), plastic tubes, etc. A solid support can comprise any of a variety of substances, including, e.g., glass, polystyrene, polyvinyl chloride, polypropylene, polyethylene, polycarbonate, dextran, nylon, amylose, natural and modified celluloses, polyacrylamides, agaroses, and magnetite. Suitable methods for immobilizing a subject antibody onto a solid support are well known and include, but are not limited to ionic, hydrophobic, covalent interactions and the like. Solid supports can be soluble or insoluble, e.g., in aqueous solution. In some embodiments, a suitable solid support is generally insoluble in an aqueous solution.

A subject antibody will in some embodiments comprise a detectable label. Suitable detectable labels include any composition detectable by spectroscopic, photochemical, biochemical, immunochemical, electrical, optical or chemical means. Detectable labels, but are not limited to, magnetic beads (e.g. Dynabeads™), fluorescent dyes (, a green fluorescent protein, a red fluorescent protein, a yellow fluorescent protein, and the like), radiolabels (e.g., ³H, ¹²⁵I, ³⁵S, ¹⁴C, or ³²P), enzymes (e.g., horse radish peroxidase, alkaline phosphatase, luciferase, and others commonly used in an enzyme-linked immunosorbent assay (ELISA)), and colorimetric labels such as colloidal gold or colored glass or plastic (e.g. polystyrene, polypropylene, latex, etc.) beads or particles. Fluorescent dyes include, e.g., isothiocyanate, texas red, cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives. Fluorescent dyes are discussed, for example, in U.S. Pat. Nos. 4,452,720, 5,227,487, and 5,543,295. One substrate for peroxidase is 3,3′,5,5′-tetramethylbenzidine (TMB).

In some embodiments, a subject antibody comprises a contrast agent or a radioisotope, where the contrast agent or radioisotope is one that is suitable for use in imaging, e.g., imaging procedures carried out on humans. Non-limiting examples of labels include radioisotope such as ¹²³I (iodine), ¹⁸F (fluorine), ⁹⁹Tc (technetium), ¹¹¹In (indium), and ⁶⁷Ga (gallium), and contrast agent such as gadolinium (Gd), dysprosium, and iron. Radioactive Gd isotopes (e.g., ¹⁵³Gd) also are available and suitable for imaging procedures in non-human mammals. A subject antibody can be labeled using standard techniques. For example, a subject antibody can be iodinated using chloramine T or 1,3,4,6-tetrachloro-3α,6α-dephenylglycouril. For fluorination, fluorine is added to a subject antibody during the synthesis by a fluoride ion displacement reaction. See, Muller-Gartner, H., TIB Tech., 16:122-130 (1998) and Saji, H., Crit. Rev. Ther. Drug Carrier Syst., 16(2):209-244 (1999) for a review of synthesis of proteins with such radioisotopes.

A subject antibody can also be labeled with a contrast agent using standard techniques. For example, a subject antibody can be labeled with Gd by conjugating low molecular weight Gd chelates such as Gd diethylene triamine pentaacetic acid (GdDTPA) or Gd tetraazacyclododecanetetraacetic (GdDOTA) to the antibody. See, Caravan et al., Chem. Rev. 99:2293-2352 (1999) and Lauffer et al., J. Magn. Reson. Imaging, 3:11-16 (1985). A subject antibody can be labeled with Gd by, for example, conjugating polylysine-Gd chelates to the antibody. See, for example, Curtet et al., Invest. Radiol., 33(10):752-761 (1998). Alternatively, a subject antibody can be labeled with Gd by incubating paramagnetic polymerized liposomes that include Gd chelator lipid with avidin and biotinylated antibody. See, for example, Sipkins et al., Nature Med., 4:623-626 (1998).

Suitable fluorescent proteins that can be linked to a subject antibody include, but are not limited to, a green fluorescent protein from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; Enhanced GFP, a red fluorescent protein; a yellow fluorescent protein; any of a variety of fluorescent and colored proteins from Anthozoan species, as described in, e.g., Matz et al. (1999) Nature Biotechnol. 17:969-973; and the like. Many such fluorescent proteins are available commercially, e.g., from Clontech, Inc.

A subject antibody will in some embodiments be linked (e.g., covalently or non-covalently linked) to a fusion partner, e.g., a ligand; an epitope tag; a peptide; a protein other than an antibody; and the like. Suitable fusion partners include peptides and polypeptides that confer enhanced stability in vivo (e.g., enhanced serum half-life); provide ease of purification, e.g., (His), e.g., 6His, and the like; provide for secretion of the fusion protein from a cell; provide an epitope tag, e.g., GST, hemagglutinin (HA; e.g., CYPYDVPDYA; SEQ ID NO: 44), FLAG (e.g., DYKDDDDK; SEQ ID NO: 45), c-myc (e.g., CEQKLISEEDL; SEQ ID NO: 46), and the like; provide a detectable signal, e.g., an enzyme that generates a detectable product (e.g., β-galactosidase, luciferase), or a protein that is itself detectable, e.g., a green fluorescent protein, a red fluorescent protein, a yellow fluorescent protein, etc.; provides for multimerization, e.g., a multimerization domain such as an Fc portion of an immunoglobulin; and the like.

The fusion protein may also include an affinity domain, including peptide sequences that can interact with a binding partner, e.g., such as one immobilized on a solid support, useful for identification or purification. Consecutive single amino acids, such as histidine, when fused to a protein, can be used for one-step purification of the fusion protein by high affinity binding to a resin column, such as nickel sepharose. Exemplary affinity domains include His5 (HHHHH) (SEQ ID NO: 44), HisX6 (HHHHHH) (SEQ ID NO: 47), C-myc (EQKLISEEDL) (SEQ ID NO: 48), Flag (DYKDDDDK) (SEQ ID NO: 49), StrepTag (WSHPQFEK) (SEQ ID NO: 50), hemagglutinin, e.g., HA Tag (YPYDVPDYA; SEQ ID NO: 51), glutathinone-S-transferase (GST), thioredoxin, cellulose binding domain, RYIRS (SEQ ID NO: 52), Phe-His-His-Thr, chitin binding domain, S-peptide, T7 peptide, SH2 domain, C-end RNA tag, WEAAAREACCRECCARA (SEQ ID NO: 53), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, 5100 proteins, parvalbumin, calbindin D9K, calbindin D28K, and calretinin, inteins, biotin, streptavidin, MyoD, leucine zipper sequences, and maltose binding protein.

A subject antibody will in some embodiments be fused to a polypeptide that binds to an endogenous blood brain barrier (BBB) receptor. Linking a subject antibody to a polypeptide that binds to an endogenous BBB receptor facilitates crossing the BBB, e.g., in a subject treatment method (see below) involving administration of a subject antibody to an individual in need thereof. Suitable polypeptides that bind to an endogenous BBB include antibodies, e.g., monoclonal antibodies, or antigen-binding fragments thereof, that specifically bind to an endogenous BBB receptor. Suitable endogenous BBB receptors include, but are not limited to, an insulin receptor, a transferrin receptor, a leptin receptor, a lipoprotein receptor, and an insulin-like growth factor receptor. See, e.g., U.S. Patent Publication No. 2009/0156498.

In some embodiments, a subject antibody comprises a polyamine modification. Polyamine modification of a subject antibody enhances permeability of the modified antibody at the BBB. A subject antibody can be modified with polyamines that are either naturally occurring or synthetic. See, for example, U.S. Pat. No. 5,670,477. Useful naturally occurring polyamines include putrescine, spermidine, spermine, 1,3-deaminopropane, norspermidine, syn-homospermidine, thermine, thermospermine, caldopentamine, homocaldopentamine, and canavalmine. Putrescine, spermidine and spermine are particularly useful. Synthetic polyamines have the empirical formula CₓH_{y}N_{z}, can be cyclic or non-cyclic, branched or unbranched, and can contain hydrocarbon chains of 3-12 carbon atoms that further include 1-6 NR or N(R)₂ moieties, wherein R is H, (C₁-C₄) alkyl, phenyl, or benzyl. Polyamines can be linked to an antibody using any standard crosslinking method.

In some embodiments, a subject antibody is modified to include a carbohydrate moiety, where the carbohydrate moiety can be covalently linked to the antibody. In some embodiments, a subject antibody is modified to include a lipid moiety, where the lipid moiety can be covalently linked to the antibody. Suitable lipid moieties include, e.g., an N-fatty acyl group such as N-lauroyl, N-oleoyl, etc.; a fatty amine such as dodecyl amine, oleoyl amine, etc.; a C3-C16 long-chain aliphatic lipid; and the like. See, e.g., U.S. Pat. No. 6,638,513. In some embodiments, a subject antibody is incorporated into a liposome.

In some embodiments, a subject anti-AABH antibody is conjugated or linked to a therapeutic and/or imaging/detectable moiety. Methods for conjugating or linking antibodies are well known in the art. Associations between antibodies and labels include any means known in the art including, but not limited to, covalent and non-covalent interactions.

In one non-limiting embodiment, a subject anti-AABH antibody can be associated with a toxin, a radionuclide, an iron-related compound, a dye, an imaging reagent, a fluorescent label or a chemotherapeutic agent that would be toxic when delivered to a cancer cell. Alternatively, a subject anti-AABH antibody can be associated with detectable label, such as a radionuclide, iron-related compound, a dye, an imaging agent or a fluorescent agent for immunodetection of target antigens.

Radiolabels and radionuclides suitable for inclusion in a subject anti-AABH antibody include gamma-emitters, positron-emitters, Auger electron-emitters, X-ray emitters and fluorescence-emitters. In some embodiments, beta-or alpha-emitters are used. Examples of radionuclides useful as toxins in radiation therapy include, without limitation: ³²P, ³³P, ⁴³K, ⁵²Fe, ⁵⁷Co, 64Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Exemplary therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu. Conditions under which a chelator will coordinate a metal are described, for example, by Gasnow et al. U.S. Pat. Nos. 4,831,175, 4,454,106 and 4,472,509. As used herein, “radionuclide” and “radiolabel” are interchangeable.

In some embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamines (cyclic or linear). In some embodiments, the agents can include DOTA and its various analogs with ¹¹¹n, ¹⁷⁷Lu, ¹⁵³Sm, ^{88/90}Y, ^{62/64/67}Cu, or ^{67/68}Ga. In some embodiments, a nanoparticle can be labeled by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

⁹⁹Tc is a particularly attractive radioisotope for diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain embodiments, a subject antibody is modified to include a chelating agent for technium.

Non-limiting examples of toxins include, for example, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, trichothecenes', Clostridium perfringens phospholipase C (PLC), bovine pancreatic ribonuclease (BPR), antiviral protein (PAP), abrin, cobra venom factor (CVF), gelonin (GEL), saporin (SAP), and viscumin.

Non-limiting examples of iron-related compounds include, for example, magnetic iron-oxide particles, ferric or ferrous particles, Fe²⁰³ and Fe³⁰⁴. Iron-related compounds and methods of labeling polypeptides, proteins and peptides can be found, for example, in U.S. Pat. Nos. 4,101,435 and 4,452,773, and U.S. published applications 20020064502 and 20020136693.

In certain embodiments, a subject antibody can be covalently or non-covalently coupled to a cytotoxin or other cell proliferation inhibiting compound, in order to localize delivery of that agent to a tumor cell. For instance, the agent can be selected from: alkylating agents, enzyme inhibitors, proliferation inhibitors, lytic agents, DNA- or RNA-synthesis inhibitors, membrane permeability modifiers, DNA metabolites, dichloroethylsulfide derivatives, protein production inhibitors, ribosome inhibitors, inducers of apoptosis, and neurotoxins.

In certain embodiments, the subject antibodies can be coupled with an agent useful in imaging tumors. Such agents include: metals; metal chelators; lanthanides; lanthanide chelators; radiometals; radiometal chelators; positron-emitting nuclei; microbubbles (for ultrasound); liposomes; molecules microencapsulated in liposomes or nanospheres; monocrystalline iron oxide nanocompounds; magnetic resonance imaging contrast agents; light absorbing, reflecting and/or scattering agents; colloidal particles; and fluorophores, such as near-infrared fluorophores. In many embodiments, such secondary functionality/moiety will be relatively large, e.g., at least 25 atomic mass units (amu) in size, and in many instances can be at least 50,100 or 250 amu in size.

In still other embodiments, the secondary functionality can be a radiosensitizing agent, e.g., a moiety that increases the sensitivity of cells to radiation. Examples of radiosensitizing agents include nitroimidazoles, metronidazole and misonidazole (see: DeVita, V. T. in Harrison's Principles of Internal Medicine, p. 68, McGraw-Hill Book Co., NY, 1983). The modified antibodies that comprise a radiosensitizing agent as the active moiety are administered and localize at the target cell. Upon exposure of the individual to radiation, the radiosensitizing agent is “excited” and causes the death of the cell.

In certain embodiments, the secondary functionality is a chelate moiety for chelating a metal, e.g., a chelator for a radiometal or paramagnetic ion. In additional embodiments, it is a chelator for a radionuclide useful for radiotherapy or imaging procedures.

There is a wide range of moieties which can serve as chelators and which can be derivatized to a subject antibody. For instance, the chelator can be a derivative of 1,4,7,10-tetraazacyclododecanetetraacetic acid (DOTA), ethylenediaminetetraacetic acid (EDTA), diethylenetriaminepentaacetic acid (DTPA) and 1-p-lsothiocyanato-benzyl-methyl-diethylenetriaminepentaacetic acid (ITC-MX). These chelators typically have groups on the side chain by which the chelator can be used for attachment to polypeptides; e.g., antibodies. Such groups include, e.g., benzylisothiocyanate, by which the DOTA, DTPA or EDTA can be coupled to, e.g., an amine group.

In one embodiment, the chelate moiety is an “NxSy” chelate moiety. As defined herein, the “NxSy chelates” include bifunctional chelators that are capable of coordinately binding a metal or radiometal and, may have N2S2 or N3S cores. Exemplary NxSy chelates are described, e.g., in Fritzberg et al. (1998) PNAS 85: 4024-29; and Weber et al. (1990) Chem. 1: 431-37; and in the references cited therein.

In some embodiments, a subject anti-AABH antibody is modified to include a chemotherapeutic agent (“chemotherapeutic”), e.g., a chemotherapeutic agent is covalently or non-covalently linked to a subject anti-AABH antibody.

Chemotherapeutic agents suitable for use in modifying a subject antibody include, for example, small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication. Examples of known cytotoxic agents suitable for use are listed, for example, in Goodman et al., “The Pharmacological Basis of Therapeutics,” Sixth Edition, A. B. Gilman et al., eds./Macmillan Publishing Co. New York, 1980.

In some embodiments, a subject anti-AABH antibody is modified to include a chemotherapeutic agent that interferes with protein synthesis. Drugs that interfere with protein synthesis include, e.g., puromycin, cycloheximide, and ribonuclease.

Most of the chemotherapeutic agents currently in use in treating cancer possess functional groups that are amenable to chemical cross-linking directly with an amine or carboxyl group of a subject antibody. For example, free amino groups are available on methotrexate, doxorubicin, daunorubicin, cytosine arabinoside, bleomycin, fludarabine, and cladribine while free carboxylic acid groups are available on methotrexate, melphalan and chlorambucil.

These functional groups, i.e., free amino groups and carboxylic acids, are targets for a variety of homobifunctional and heterobifunctional chemical cross-linking agents which can crosslink these drugs directly to a free amino group of a subject antibody.

Chemotherapeutic agents contemplated for modification of a subject antibody also include other chemotherapeutic drugs that are commercially available. Merely to illustrate, the chemotherapeutic can be an inhibitor of chromatin function, a replication inhibitor, an inhibitor of cell division, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and/or a DNA repair inhibitor.

Chemotherapeutic agents may be categorized by their mechanism of action into, for example, the following groups: anti-metabolites/anti-cancer agents, such as pyrimidine analogs (fluorouracil, azaribine, floxuridine, capecitabine, and cytarabine) and purine analogs (mercaptopurine and thioguanine), folate antagonists and related inhibitors antiproliferative/antimitotic agents including natural products such as vinca alkaloid (vinblastine, vincristine), and inhibitors of microtubule function such as taxane (paclitaxel, docetaxel), vinblastin, nocodazole, epothilones and navelbine, epidipodophyllotoxins (etoposide, teniposide), DNA damaging agents (actinomycin, amsacrine, busulfan, carboplatin, chlorambucil, cisplatin, cyclophosphamide, cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, triethylenethiophosphoramide and etoposide; antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (cyclophosphamide and analogs, mechlorethamine, melphalan, chlorambucil, uracil mustard hexamethylmelamine and thiotepa), alkyl nitrosoureas (BCNU) and analogs, streptozocin, trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones (e.g., estrogen (diethylstilbestrol and ethinyl estradiol) and androgen (testosterone propionate and fluoxymesterone), hormone analogs (e.g., tamoxifen, goserelin, bicalutamide, nilutamide), aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (e.g., breveldin); immunosuppressives, tacrolimus sirolimus azathioprine, mycophenolate; compounds (TNP-470, genistein), growth factor inhibitors (vascular endothelial growth factor inhibitors, fibroblast growth factor inhibitors); angiotensin receptor blocker, nitric oxide donors; anti-sense oligonucleotides; antibodies (trastuzumab, rituximab); cell cycle inhibitors and differentiation inducers (tretinoin), topoisomerase inhibitors (doxorubicin, adriamycin, daunorubicin, dactinomycin, eniposide, epirubicin, etoposide, idarubicin, irinotecan and mitoxantrone, topotecan, irinotecan), corticosteroids (cortisone, dexamethasone, hydrocortisone, methylpednisolone, prednisone, and prenisolone); growth factor signal transduction kinase inhibitors; dysfunction inducers, toxins (e.g., Cholera toxin, ricin, Pseudomonas exotoxin, Bordetella pertussis adenylate cyclase toxin, or diphtheria toxin), caspase activators; and chromatin remodeling agents. Preferred dosages of the chemotherapeutic agents are consistent with currently prescribed dosages.

As used herein, the terms “nucleic acid damaging treatment” and “nucleic acid damaging agent” refer to any treatment regimen that directly or indirectly damages nucleic acid (e.g., DNA, cDNA, genomic DNA, mRNA, tRNA or rRNA). Examples of such agents include alkylating agents, nitrosoureas, anti-metabolites, plant alkaloids, plant extracts and radioisotopes. Examples of nucleic acid damaging agents also include nucleic acid damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′,2′-difluoro-2′-deoxycytidine (dFdC), purine antimetabolites (mercaptopurine, azathiopurine, thioguanine), gemcitabine hydrochloride (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubibcin (Cerubicine), idarubicin (Idamycin) and epirubicin (Ellence), anthracyclin analogues such as mitoxantrone, actinomycin D, non-intercalating topoisomerase inhibitors such as epipodophyllotoxins (etoposide=VP16, teniposide=VM-26), podophylotoxin, bleomycin (Bleo), pepleomycin, compounds that form adducts with nucleic acid including platinum derivatives (e.g., cisplatin (CDDP), trans analogue of cisplatin, carboplatin, iproplatin, tetraplatin and oxaliplatin), camptothecin, topotecan, irinotecan (CPT-11), and SN-38. Specific examples of nucleic acid damaging treatments include radiation (e.g., focused microwaves, ultraviolet (UV), infrared (IR), or alpha-, beta- or gamma-radiation) and environmental shock (e.g., hyperthermia).

As used herein, the terms “anti-proliferative treatment” and “anti-proliferative agent” means any treatment regimen that directly or indirectly inhibits proliferation of a cell, virus, bacteria or other unicellular or multicellular organism regardless of whether or not the treatment or agent damages nucleic acid. Particular examples of anti-proliferative agents are anti-tumor and anti-viral drugs, which inhibit cell proliferation or virus proliferation or replication. Examples include, inter alia, cyclophosphamide, azathioprine, cyclosporin A, prednisolone, melphalan, chlorambucil, mechlorethamine, busulphan, methotrexate, 6-mercaptopurine, thioguanine, cytosine arabinoside, taxol, vinblastine, vincristine, doxorubicin, actinomycin D, mithramycin, carmustine, lomustine, semustine, streptozotocin, hydroxyurea, cisplatin, mitotane, procarbazine, dacarbazine and dibromomannitol. Anti-proliferative agents that cause nucleic acid replication errors or inhibit nucleic acid replication are those such as nucleoside and nucleotide analogues (e.g., AZT or 5-AZC).

In another embodiment, a subject anti-AABH antibody can be conjugated to a “receptor” (such as biotin or streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionuclide).

### VI. Kits

Provided herein are kits. Kits can be provided in a container, such as a box or bag, e.g., adapted for shipping by, for example, sealing and/or providing slots to accommodate articles, such as vials, microtiter plates, etc. Kits can comprise an antibody or a conjugated antibody as disclosed herein, and any of: a second antibody, e.g., for performing a sandwich assay, reagents for performing immunoassays and assay plates, such as microwell plates. Compounds can be provided in sub-containers, such as vials or tubes.

In one embodiment, the kit comprises the following items: (1) A solid support to which is attached a first antibody against AABH; (2) a second anti-AABH antibody conjugated with biotin; (3) streptavidin-conjugated horse radish peroxidase; and (4) a chromogenic substrate for horse radish peroxidase, such as 3,3′,5,5′-Tetramethylbenzidine (TMB), 3,3′-Diaminobenzidine (DAB) or 2,2′-azino-bis(3-ethylbenzothiazoline-6-sulphonic acid) (ABTS).

### VII. Methods of Detecting AABH, AABH Epitopes and AABH Fragments

Provided herein are methods of detecting AABH, AABH fragments or polypeptides comprising AABH epitopes in a biological sample. Such methods are useful for diagnosing conditions associated with overexpression of AABH. The methods involve contacting a biological sample with an antibody of this disclosure and detecting binding between the antibody and AABH, fragment or a polypeptide epitope thereof. In certain embodiments, the antibody is affixed to a substrate (e.g., a solid support) for convenience in conducting the assay and detecting the antibody-antigen complex. Exemplary substrates include a culture dish, an assay plate, an assay well, a nitrocellulose membrane, a bead, a dipstick, and a component of an elution column.

A. Immunoassays

Any immunoassay method known in the art can be used for detection of AABH. In one embodiment, the immunoassay is a competitive immunoassay, in which an unlabeled analyte in a sample competes with a labeled analyte to bind to an antibody. In another embodiment, the immunoassay is a noncompetitive immunoassay in which an analyte binds with an antibody which may be labeled or unlabeled. Noncompetitive immunoassays include both one-site immunoassays and two-site immunoassays. In a one-site immunoassay an analyte binds with an antibody, and binding is detected through a label or some other method. In a two-site immunoassay, an analyte is bound to an unlabeled antibody and then a labeled antibody is bound to the analyte and detected. Two-site immunoassays are typically referred to as “sandwich immunoassays”.

One form of immunoassay using a detectable label is the enzyme-linked immunosorbent assay (“ELISA”). ELISA uses an antibody labeled with an enzyme which catalyzes a reaction producing a detectable product. In some cases, an antibody specific for the target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Streptavidin-biotin interaction) and detected using a labeled antibody (or other targeting moiety). Several permutations of the original ELISA methods have been developed and are known in the art (see Lequin (2005) Clin. Chem. 51:2415-18 for a review).

Label-free immunoassays include, for example, use of surface plasmon resonance or interferometry. (See, for example, U.S. Pat. No. 5,641,640 (Hanning) and WO 2009/039466 (Bornhop).) The Kd, Kon, and Koff can also be determined using surface plasmon resonance (SPR), e.g., as measured by using a Biacore T100 system. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When the surface is exposed to light of a given frequency, the changes to the optical reflectivity of the gold indicate binding, and the kinetics of binding. Binding affinity can also be determined by anchoring a biotinylated interactant to a streptavidin (SA) sensor chip. The other interactant is then contacted with the chip and detected, e.g., as described in Abdessamad et al. (2002) Nuc. Acids Res. 30:e45.

In another embodiment, the immunoassay is a lateral flow immunoassay. In one form of the lateral flow assay, a sample comprising the analyte is deposited on a test strip, e.g., nitrocellulose. The analyte moves via capillary action up the strip to a position containing, e.g., a detectably labeled binding agent such as an antibody, directed to the analyte. The detectable agent can be, e.g., colloidal gold or latex beads. The analyte is captured and continued to move up the strip to a detection position, where is immobilized a second capture agent against the analyte. The complex is captured here, and detected. Upstream of the detection position is a control position comprising immobilized capture agent that binds to the labeled binding agent. In a competitive format, the unlabeled analyte moves to a position containing labeled analyte. At the detection position, lack of detectable label indicates binding by the unlabeled analyte.

One example of a competitive immunoassay is a radioimmunoassay. In a radioimmunoassay, a radioactively labeled antigen competes for binding to an antibody with antigens in a sample. A measure of radioactive antigen bound inversely reflects the amount of analyte in the sample.

B. Detectable Labels

Detectable labels for attachment to antibodies can be any of those known in the art and as described herein. Examples are provided in the following references: Armstrong et al., Diagnostic Imaging, 5^{th} Ed., Blackwell Publishing (2004); Torchilin, V. P., Ed., Targeted Delivery of Imaging Agents, CRC Press (1995); Vallabhajosula, S., Molecular Imaging: Radiopharmaceuticals for PET and SPECT, Springer (2009); C. Kessler, ed. Nonradioacticve Labeling and Detection of Biomolecules, Springer-Verlag, 1992; and G. C. Howard, ed., Methods in Nonradioactive Detection, Appleton & Lange, 1993. A detectable label can be detected in a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

In some embodiments, the detectable label can include optical agents such as fluorescent agents, phosphorescent agents, chemiluminescent agents, and the like. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)).

The label can also be a radioisotope, e.g., radionuclides that emit gamma rays, positrons, β and alpha particles, and X-rays, as described.

In some embodiments, the detectable label can be an enzyme that produces a detectable signal when an enzyme substrate is put into contact with it. Such labels are used in ELISA assays. The enzyme can be, for example, urease, β-galactosidase, β-glucuronidase, alkaline phosphatase, (horseradish) hydrogen peroxidase or glucose oxidase. Secondary binding ligands include, e.g., biotin and avidin or streptavidin compounds as known in the art.

In some embodiments, the labeled antibody can be further associated to a composition that improves stability in vivo, e.g. PEG or a nanoparticle such as a liposome.

C. Methods of Labeling

Techniques for conjugating detectable labels or drugs to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

Typically, the antibody is attached to the detectable label in an area that does not interfere with binding to the epitope. Thus, in some cases, the detectable label is attached to the constant region, or outside the CDRs in the variable region. One of skill in the art will recognize that the detectable label can be located elsewhere on the antibody, and the position of the detectable label can be adjusted accordingly. In some embodiments, the ability of the antibody to associate with the epitope is compared before and after attachment to the detectable label to ensure that the attachment does not unduly disrupt binding.

In some embodiments, the antibody can be associated with an additional targeting moiety. For example, an antibody fragment, peptide, or aptamer that binds a different site on the target molecule or target cell can be conjugated to the antibody to optimize target binding, e.g., to a cancer cell.

D. Method of Attaching Antibody to Microtiter Plate

Antibodies can be attached to a surface of a microtiter plate by the following exemplary method. Dilute unlabeled capture antibody to a final concentration of 1-10 μg/mL using PBS or carbonate/bicarbonate buffer (pH7.4). Transfer 50-100 μL per well to ELISA microplate. Seal the plate and incubate overnight at 4° C. Wash the plate three times with PBS/Tween by using a squirt bottle, multi-channel pipettor or automatic plate washer. Tap the plate on paper towels after the last wash to remove residual wash solution. Blocking the Plate: Block the Plate with Blocking Buffer at 200 μl/well. Seal the plate and incubate at 37° C. for 2 hr or at 4° C. overnight.

### VIII. Methods of Diagnosing Conditions Associated with Over-Expression of AABH

Methods for detecting AABH, AABH fragments and polypeptides comprising AABH polypeptide epitopes are useful for diagnosing conditions associated with overexpression of AABH, in particular, cancer. AABH expression is upregulated at the gene expression level in cancer cells and this increased expression serves as the basis for a highly sensitive test for diagnosis and monitoring of disease as well as the measurement of drug efficacy. This test is capable of detecting one cancer cell in a background of 50,000 normal cells and thus has potential application for the measurement of minimal residual disease in various blood cancers (e.g., any cancer listed in Table 1), as well as recurrence of disease.

The antibodies described herein specifically bind AABH and AABH-expressing cells. The monoclonal antibodies provided herein can thus be used for in vitro and in vivo diagnostic assays to detect AABH-expressing cells. For example, a sample (e.g., blood sample or tissue biopsy) can be obtained from a patient and contacted with an AABH antibody, and the presence of an AABH expressing cell in the patient sample can be determined by detecting antibody binding. Antibody binding can be detected by any method described herein.

In some embodiments, the anti-AABH antibody is contacted with a biological sample from an individual having or suspected of having a condition associated with overexpression of AABH, such as cancer, and antibody binding to a cell in the sample is determined, wherein higher or lower than normal antibody binding indicates that the individual has a condition associated with overexpression of AABH.

The biological sample to be tested can be a biopsy. Biopsies are typically performed to obtain samples from tissues, i.e., non-fluid cell types. The biopsy technique applied will depend on the tissue type to be evaluated (e.g., breast, skin, esophagus, stomach, colon, prostate, kidney, lung, bladder, lymph node, liver, bone marrow, airway or lung). In the case of a cancer the technique will also depend on the size and type of the tumor (e.g., solid, suspended, or blood), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

In some embodiments detection of AABH an AABH fragment or an AABH polypeptide epitope in a sample above a control amount indicates the presence of the condition, for example, the presence of cancer. The test can be selected such that cutoffs are set to provide the requisite level of sensitivity, specificity, accuracy and/or positive predictive value. For example, cutoff values can be selected so that the diagnostic test has at least any of 70%, 75%, 80%, 85%, 90%, 95%, 98%, 99%, 99.5%, or 99.9% sensitivity, specificity, accuracy and/or positive predictive value.

In certain embodiments, a cutoff value is obtained by measuring AABH levels in a plurality of cancerous and non-cancerous reference samples, e.g., at least 10, 20, 50, 100 or 200 samples of each type. A cut-off can be established with respect to a measure of central tendency, such as mean, median or mode. A measure of deviation from this measure of central tendency can be used to set the cut off. For example, the cut off can be set based on variance or standard deviation. For example, the cut off can be based on Z score, that is, a number of standard deviations above a mean of normal samples, for example one standard deviation, two standard deviations, three standard deviations or four standard deviations. These numbers, in turn, can be set to provide a desired level of sensitivity and/or specificity. For example, it may be determined that a Z score above 1 provides a specificity of 90%.

It has been found that in a blood sample, e.g., whole blood, plasma or serum, an amount of 0.3 ng/ml of AABH provides a cut off that is highly sensitive and specific for cancer. Increasing the cut off decreases the sensitivity of the assay and increases specificity. Decreasing the cut off increases the sensitivity of the assay and decreases specificity. A cut off of 2.5 ng/ml is expected to produce a test with about 90% specificity. Alternatively, a cut off of about 4.0 ng/ml decreases sensitivity to about 90%. Accordingly, one can diagnose cancer in a subject is the amount of AABH in a sample, in particular, a blood sample, is above any of 0.20 ng/ml, 0.21 ng/ml, 0.22 ng/ml, 0.23 ng/ml, 0.24 ng/ml, 0.25 ng/ml, 0.26 ng/ml, 0.27 ng/ml, 0.28 ng/ml, 0.29 ng/ml, 0.30 ng/ml, 0.31 ng/ml, 0.32 ng/ml, 0.33 ng/ml, 0.34 ng/ml, 0.35 ng/ml, 0.36 ng/ml, 0.37 ng/ml, 0.37 ng/ml, 0.39 ng/ml, 0.40 ng/ml, 0.41 ng/ml, 0.42 ng/ml, 0.43 ng/ml, 0.44 ng/ml, 0.45 ng/ml, 0.46 ng/ml, 0.47 ng/ml, 0.47 ng/ml, 0.49 ng/ml, 0.50 ng/ml, 0.50 ng/ml, 0.60 ng/ml, 0.70 ng/ml, 0.80 ng/ml, 0.90 ng/ml, 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/m or 2.0 ng/ml.

In some embodiments, the percentage of AABH-expressing cells in the sample is determined and compared to a control, e.g., a sample from an individual or group of individuals that are known to have a condition associated with overexpression of AABH (positive control) or from an individual or group of individuals that are known not to have a condition associated with overexpression of AABH (normal, non-disease, or negative control). In some embodiments, the control is a standard range of AABH expression established for a given tissue. A higher or lower than normal percentage of AABH expressing cells, or higher or lower expression level of AABH per cell, indicates that the individual has a condition associated with overexpression of AABH.

In some embodiments, a labeled anti-AABH antibody can be provided (administered) to an individual to determine the applicability of an intended therapy. For example, a labeled antibody may be used to detect AABH density within a diseased area, where the density is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.).

In some embodiments, labeled antibodies specific for AABH as described herein can be further associated with a therapeutic compound, e.g., to form a “theranostic” composition. For example, an anti-AABH antibody described herein can be linked (directly or indirectly) to both a detectable label and a therapeutic agent, e.g., a cytotoxic agent, to kill AABH-expressing cancer cells. In some embodiments, a labeled antibody specific for AABH is used for diagnosis and/or localization of an AABH expressing cancer cell, and the AABH expressing cancer cell is then targeted with a separate therapeutic antibody specific for AABH. In some embodiments, the diagnostic antibody that is specific for AABH is one that is not internalized into AABH expressing cells at a high rate or percentage. In some embodiments, the therapeutic antibody specific for AABH is an antibody that inhibits proliferation of AABH expressing cells upon crosslinking or multimerization.

In another aspect, provided herein in is a method comprising recommending or prescribing to a subject determined to have cancer based, at least in part, on having an AABH level above a diagnostic cut off, or to a health care provider providing care for the subject, a therapeutic intervention to treat the cancer. The determination can be my measuring AABH by a method as disclosed herein.

In another aspect provided herein in is a method comprising administering to a subject determined to have cancer based, at least in part, on having an AABH level above a diagnostic cut off, a therapeutic intervention to treat the cancer. The determination can be my measuring AABH by a method as disclosed herein. The therapeutic intervention can include, for example, one or more of chemotherapy, radiation therapy, surgery, immunotherapy, hormone therapy or stem cell therapy. Immunotherapy modalities include, for example, antibody-drug conjugates, CAR (chimeric antigen receptor) T-cells and checkpoint inhibitors.

In another aspect provided herein is a method for determining effectiveness of a therapeutic intervention in treating a cancer comprising: (a) determining, in each subject in a population comprising a plurality of subjects, an initial relative amount of AABH in a biological sample from the subject; (b) after determining, administering the therapeutic intervention to the subjects; (c) after administering, determining, in each subject individual in the population, a subsequent relative amount of AABH; and (d) based on the initial and subsequent amounts in the population, that the therapeutic intervention is effective if the subsequent amounts exhibit a statistically significant decrease compared with the initial amounts, or that the therapeutic intervention is not effective if the subsequent amounts do not exhibit a statistically significant decrease compared with the initial amounts. In one embodiment the therapeutic intervention comprises administration of a drug or combination of drugs. In another embodiment the biological sample comprises blood. In another embodiment the population comprises at least 50, at least 100 or at least 200 subjects, wherein at least 20%, at least 35%, at least 50%, or at least 66% of the subjects initially have elevated amounts of AABH. In another embodiment at least 20%, at least 35%, at least 50%, or at least 66% of the subjects initially have a diagnosis of a cancer. In another embodiment statistical significance is determined, at least in part, based on a control group of subjects to whom the therapeutic intervention is not administered, or is administered in different amounts or different routes or at different rates or times.

In another aspect provided herein is a method for qualifying subjects for a clinical trial of a therapeutic intervention for the treatment or prevention of a cancer comprising: a) determining that a subject has an amount of AABH in a biological sample that are above a threshold amount; and b) enrolling the subject in the clinical trial of a potentially therapeutic intervention for said cancer. In one embodiment the threshold amount is based on a measure of deviance from a measure of central tendency consistent with the subject having a condition selected from a cancer or increased risk of developing a cancer.

In another aspect provided herein is a method of monitoring progress of a subject on a therapeutic intervention for a cancer comprising: (a) determining, in the subject, an initial amount of AABH in a biological sample from the subject; (b) administering the therapeutic intervention to the subject after the determination; (c), determining, in the subject, after administering the therapeutic intervention a subsequent amount of AABH in a biological sample from the subject; and (d) based on the relative amounts, determining that the subject is responding positively to the therapeutic intervention if the amounts exhibit a statistically significant decrease in the initial and subsequent amounts, or that the subject is not responding positively to the therapeutic intervention if the amounts do not exhibit a statistically significant decrease in the initial and subsequent amounts.

### IX. Therapeutic Applications

AABH is overexpressed in malignant cells, including, without limitation, lung cancer, breast cancer, prostate cancer and colon cancer. Accordingly, in another aspect, this disclosure provides methods of treating cancer and pre-cancerous states. In one embodiment, the methods involve targeting cells expressing AABH, and polypeptides having amino acid sequences comprising, consisting essentially of, or consisting of the amino acid sequences of the AABH epitopes of this disclosure (and functional equivalents) with the antibodies disclosed herein, e.g., in the form of antibody-drug conjugates. In another embodiment, the methods involve evoking an immune response against cells expressing AABH by immunizing a subject with the polypeptide epitopes and chimeric polyepitopes (and functional equivalents) of this disclosure.

A. Antibody-Drug Conjugates

Antibody-drug conjugates (“ADCs”) comprise an antibody against a target antigen conjugated to a drug, typically through a linker. In the present case, the antibody can be an antibody of disclosure that binds AABH or a polypeptide comprising a polypeptide epitope of AABH or a functional equivalent thereof. For purposes of cancer therapy, the drug is a chemotherapeutic or cytotoxic agent.

1. Chemotherapeutic and Cytotoxic Agents

Anti-AABH antibodies can inhibit cancer cell growth (proliferation), and thus can be considered chemotherapeutic agents. The following disclosure provides examples of various chemotherapeutic and cytotoxic agents that can be linked to an anti-AABH antibody for targeting AABH-expressing cells.

A chemotherapeutic (anti-cancer) agent can be any agent capable of reducing cancer growth, interfering with cancer cell replication, directly or indirectly killing cancer cells, reducing metastasis, reducing tumor blood supply, etc. Chemotherapeutic agents thus include cytotoxic agents. Cytotoxic agents include but are not limited to ricin, diphtheria toxin, saporin, taxanes, vinca alkaloids, anthracycline, and platinum-based agents. Classes of chemotherapeutic agents include but are not limited to alkylating agents, antimetabolites, e.g., methotrexate, plant alkaloids, e.g., vincristine, and antitumor antibiotics such as anthracyclines, e.g., doxorubicin as well as miscellaneous drugs that do not fall into a particular class such as hydroxyurea. Platinum-based drugs, exemplified by cisplatin and oxaliplatin, represent a major class of chemotherapeutics. These drugs bind to DNA and interfere with replication. Taxanes, exemplified by taxol, represent another major class of chemotherapeutics. These compounds act by interfering with cytoskeletal and spindle formation to inhibit cell division, and thereby prevent growth of rapidly dividing cancer cells. Other chemotherapeutic drugs include those used in hormonal therapy. Chemotherapeutics also include agents that inhibit tubulin assembly or polymerization such as maytansine, mertansine, and auristatin. Chemotherapeutic agents also include DNA damaging agents such as calicheamicin.

Chemotherapeutic agents can include maytansinoid, auristatin, dolastatin, tubulysin, cryptophycin, pyrrolobenzodiazepine (PBD) dimer, indolinobenzodiazepine dimer, alpha-amanitin, trichothene, SN-38, duocarmycin, CC1065, calicheamincin, an enediyne antibioatic, taxane, doxorubicin derivatives, anthracycline and stereoisomers, azanofide, isosteres, analogs or derivatives thereof.

A. Methods of Forming Therapeutic Compositions

Antibodies can be attached to a drug, a detectable label or nanocarrier using a variety of known cross-linking agents. Methods for covalent or non-covalent attachment of polypeptides are well known in the art. Such methods may include, but are not limited to, use of chemical cross-linkers, photoactivated cross-linkers and/or bifunctional cross-linking reagents. Exemplary methods for cross-linking molecules are disclosed in U.S. Pat. Nos. 5,603,872 and 5,401,511. Non-limiting examples of cross-linking reagents include glutaraldehyde, bifunctional oxirane, ethylene glycol diglycidyl ether, carbodiimides such as 1-ethyl-3-(3-dimethylaminopropyl) carbodiimide or dicyclohexylcarbodiimide, bisimidates, dinitrobenzene, N-hydroxysuccinimide ester of suberic acid, disuccinimidyl tartarate, dimethyl-3,3′-dithio-bispropionimidate, azidoglyoxal, N-succinimidyl-3-(2-pyridyldithio)propionate and 4-(bromoadminoethyl)-2-nitrophenylazide. Agents can be conjugated through cysteine residues in the antibody, which can be naturally occurring or artificially introduced. Linkers can comprise a reactive group at one end which binds to the antibody, a spacer comprising a plurality of polyethylene units and one or more amino acids attached through a group to the drug. See, e.g., U.S. Pat. No. 9,527,871 for exemplary linker chemistries.

In some embodiments, the antibody is linked to a stabilizing moiety such as PEG, or a liposome or other nanocarrier. U.S. Pat. Nos. 4,732,863 and 7,892,554 and Chattopadhyay et al. (2010) Mol Pharm 7:2194 describe methods for attaching the selected antibody to PEG, PEG derivatives, and nanoparticles (e.g., liposomes). Liposomes containing phosphatidyl-ethanolamine (PE) can be prepared by established procedures as described herein. The inclusion of PE provides an active functional site on the liposomal surface for attachment.

The antibody conjugate can also be formulated to provide more than one active compound, e.g., additional chemotherapeutic or cytotoxic agents, cytokines, or growth inhibitory agents. The active ingredients may also prepared as sustained-release preparations (e.g., semi-permeable matrices of solid hydrophobic polymers (e.g., polyesters, hydrogels (for example, poly (2-hydroxyethyl-methacrylate), or poly (vinylalcohol)), polylactides. The antibodies and immunoconjugates can be entrapped in a nanoparticle prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

Preparation of nanocarriers, such as an antibody targeted liposome, polymeric nanoparticle, or extended shelf-life liposome, is described, e.g., in U.S. Pat. Nos. 6,465,188, 7,122,202, 7,462,603 and 7,550,441.

B. Methods of Administration

In some embodiments, a method of treatment comprises administering to an individual an effective amount of an antibody-drug conjugate of this disclosure. In other embodiments, a use of an effective amount of an antibody-drug conjugate of this disclosure is provided to treat an individual. In further embodiments, an effective amount of an antibody-drug conjugate of this disclosure for use in the treatment of an individual in provided. In some embodiments, the individual has been diagnosed with cancer. In some embodiments, the individual is receiving or has received cancer therapy, e.g., surgery, radiotherapy, or chemotherapy. In some embodiments, the individual has been diagnosed, but the cancer is in remission.

In some embodiments, the invention can include an antibody or antibody-targeted composition and a physiologically (i.e., pharmaceutically) acceptable carrier. The term “carrier” refers to a typically inert substance used as a diluent or vehicle for a diagnostic or therapeutic agent. The term also encompasses a typically inert substance that imparts cohesive qualities to the composition. Physiologically acceptable carriers can be liquid, e.g., physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Since physiologically acceptable carriers are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

The compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized antibody compositions.

In some embodiments, the anti-AABH conjugate includes a liposome. In some embodiments, the method further comprises monitoring the individual for progression of the cancer. In some embodiments, the dose of the anti-AABH conjugate for each administration is determined based on the therapeutic progress of the individual, e.g., where a higher dose of chemotherapeutic is administered if the individual is not responding sufficiently to therapy.

Dosage forms can be prepared for mucosal (e.g., nasal, sublingual, vaginal, buccal, or rectal), parenteral (e.g., subcutaneous, intravenous, intramuscular, or intraarterial injection, either bolus or infusion), oral, or transdermal administration to a patient. Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Injectable (e.g., intravenous) compositions can comprise a solution of the antibody or antibody-targeted composition suspended in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) will be used. The compositions can contain pharmaceutically acceptable auxiliary substances to approximate physiological conditions, such as pH adjusting and buffering agents, tonicity adjusting agents, wetting agents, e.g., sodium acetate, sodium lactate, sodium chloride, potassium chloride, calcium chloride, sorbitan monolaurate, triethanolamine oleate, etc. In some embodiments, the antibody-targeted composition can be formulated in a kit for intravenous administration.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, topically, intraperitoneally, intravesically, or intrathecally. Parenteral administration and intravenous administration are the preferred methods of administration. The formulations of targeted compositions can be presented in unit-dose or multi-dose sealed containers, such as ampoules and vials.

The targeted delivery composition of choice, alone or in combination with other suitable components, can be made into aerosol formulations (“nebulized”) to be administered via inhalation. Aerosol formulations can be placed into pressurized acceptable propellants, such as dichlorodifluoromethane, propane, and nitrogen.

The pharmaceutical preparation can be packaged or prepared in unit dosage form. In such form, the preparation is subdivided into unit doses containing appropriate quantities of the active component, e.g., according to the dose of the therapeutic agent or concentration of antibody. The unit dosage form can be a packaged preparation, the package containing discrete quantities of preparation. The composition can, if desired, also contain other compatible therapeutic agents.

The antibody (or antibody-targeted composition) can be administered by injection or infusion through any suitable route including but not limited to intravenous, subcutaneous, intramuscular or intraperitoneal routes. An example of administration of a pharmaceutical composition includes storing the antibody at 10 mg/ml in sterile isotonic aqueous saline solution for injection at 4° C., and diluting it in either 100 ml or 200 ml 0.9% sodium chloride for injection prior to administration to the patient. The antibody is administered by intravenous infusion over the course of 1 hour at a dose of between 0.2 and 10 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.1 mg/kg body weight to about 25 mg/kg body weight or in the range 1 mg-2 g per patient. In some cases, the dose is in the range 1-100 mg/kg, or approximately 50 mg-8000 mg/patient. The dose may be repeated at an appropriate frequency which may be in the range once per day to once every three months, depending on the pharmacokinetics of the antibody (e.g., half-life of the antibody in the circulation) and the pharmacodynamic response (e.g., the duration of the therapeutic effect of the antibody). In some embodiments, the in vivo half-life is between about 7 and about 25 days and antibody dosing is repeated between once per week and once every 3 months.

Administration can be periodic. Depending on the route of administration, the dose can be administered, e.g., once every 1, 3, 5, 7, 10, 14, 21, or 28 days or longer (e.g., once every 2, 3, 4, or 6 months). In some cases, administration is more frequent, e.g., 2 or 3 times per day. The patient can be monitored to adjust the dosage and frequency of administration depending on therapeutic progress and any adverse side effects, as will be recognized by one of skill in the art.

In some embodiments, additional administration is dependent on patient progress, e.g., the patient is monitored between administrations. For example, after the first administration or round of administrations, the patient can be monitored for rate of tumor growth, recurrence (e.g., in the case of a post-surgical patient), or general disease-related symptoms such as weakness, pain, nausea, etc.

In therapeutic use for the treatment of cancer, an antibody-targeted composition (e.g., including a therapeutic and/or diagnostic agent) can be administered at the initial dosage of about 0.001 mg/kg to about 1000 mg/kg daily and adjusted over time. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosage is varied depending upon the requirements of the patient, the severity of the condition being treated, and the targeted composition being employed. For example, dosages can be empirically determined considering the type and stage of cancer diagnosed in a particular subject. The dose administered to a patient, in the context of the present invention, should be sufficient to affect a beneficial therapeutic response in the patient over time. The size of the dose will also be determined by the existence, nature, and extent of any adverse side-effects that accompany the administration of a particular targeted composition in a particular patient, as will be recognized by the skilled practitioner.

### Example

Animals were inoculated with one of two polypeptide fragments of AABH. These fragments had the following amino acid sequences:

AABH Fragment 1:
(SEQ ID NO: 38)
LDAAEK LRKRGKIEEA VNAFKELVRK YPQSPRARYG KAQCEDDLAE
KRRSNEVLRG AIETYQEVAS LPDVPADLLK LSLKRRSDRQ
QFLGHMRGSL LTLQRLVQLF PNDTSLKNDL GVGYLLIGDN
DNAKKVYEEV LSVTPNDGFA KVHYGFILKA QNKIAESIPY
LKEGIESGDP GTDDGRFYFH LGDAMQRVGN KEAYKWYELG
HKRGHFASVW Q RSLYNVNGL KAQPWWTPKE TGYTELVKSL
ERNWKLIRDE GLAVMDKAKG LFLPEDENLR EKGDWSQFTL
WQQGRRNENA CKGAPKTCTL LEKFPETTGC RRGQIKYSIM
HPGTHVWPHT GPTNCRLRMH LGLVIPKEGC KIRCANETRT
WEEGKVLIFD DSFEHEVWQD ASSFRLIFIV DVWHPELTPQ
QRRSLPAI;
and
AABH Fragment 2:
(SEQ ID NO: 39)
ASSFRLIFIV DVWHPELTPQ QRRSLPAI.

Hybridomas were produced and monoclonal antibodies produced were tested for ability to bind AABH. One hybridoma, resulting from inoculation with AABH Fragment 1 was selected for further testing. The monoclonal antibody was used to measure amounts of AABH in test samples.

The assay can proceed as follows:

Sample Collection and Storage:

For serum, allow sample to clot and remove clot, e.g., by centrifiguation. For plasma, collect in EDTA or heparin and centrifuge before use. Samples may be stored frozen before use.

Assay Procedure:

Provide wells coated with anti-AABH antibody. Perform assay in triplicate with blanks and dilution standards as control. Incubate wells, sealed for 2 hours at 37° C.

Remove the liquid of each well, without washing and add detection reagent, which can comprise a labeled AABH antibody, e.g., coupled to horse radish peroxidase. Incubate, sealed for 1 hour at 37° C. Remove liquid from the wells and wash, for example, three times.

Add substrate, e.g., TMB, to wells. Incubate, sealed for 10-20 minutes at 37° C.

Add Stop Solution (sulfuric acid) to each well.

Read wells with plate reader.

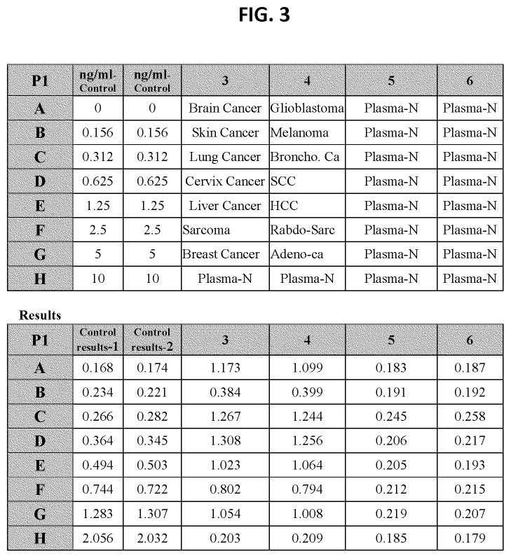

Referring to FIG. 3, top panel, samples included control samples into which AABH was spiked into normal plasma in the amounts indicated (1A-1H and 2A-2H). Plasma of 32 test subjects was also tested for amounts of AABH (ng/ml). Test subjects included both those with cancer (subjects 3A-3G and 4A-4G) and those without cancer (3H, 4H, 5A-5H and 6A-6H). Normal subjects are designated “Plasma-N”. Cancer subjects are designated with the particular cancer. These included: brain, skin, lung, cervix, liver, sarcoma, breast, glioblastoma, melanoma, bronchial carcinoma, squamous cell carcinoma (SCC), hepatocellular carcinoma (HCC), rhabdomyosarcoma, and adenocarcinoma. Referring to FIG. 3, bottom panel, the amounts of AABH in ng/ml of each sample is identified. These results show that all control samples spiked with less than 0.3 ng/ml of AABH, and all non-cancer subjects, the amount of AABH measured was less than 0.3 ng/ml. In contrast, all control samples spiked with more than 0.3 ng/ml AABH and all cancer subjects, the amount of AABH measured was greater than 0.3 ng/ml. All cancer patients had amounts of AABH of at least 0.384 ng/ml. All non-cancer patients had amounts of AABH of 0.258 ng/ml or below.

All publications and patent applications mentioned in this specification are herein incorporated by reference to the same extent as if each individual publication or patent application was specifically and individually indicated to be incorporated by reference.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of making a hybridoma that produces antibodies against aspartyl (asparaginyl) β-hydroxylase (“AABH”) comprising:
a) immunizing an animal with a polypeptide to elicit an immune response, wherein the polypeptide is selected from:
(i) a polypeptide having an amino acid sequence consisting of, or consisting essentially of:
SEQ ID NO: 1
CRRGQIKYS;
SEQ ID NO: 2
GPTNCRLRMHLGLVI;
SEQ ID NO: 3
RT WEEGKVLIFD;
SEQ ID NO: 4
WQD ASSFRLIFI;
SEQ ID NO: 5
LS GTSFFTWFMVIALLGVWTSV;
SEQ ID NO: 6
VYEEVLSVTPNDGFAKVHYGFILKAQNK;
SEQ ID NO: 7
FASVWQRSLYNVNGLK;
SEQ ID NO: 8
GLSGTSFFTWFMVIALLGVWTSVA;
SEQ ID NO: 9
GDGDFDVDDAKVLLGLKERSTSEPAV;
SEQ ID NO: 10
IEEAVNAFKELVRKYPQSPRARYGKAQC;
SEQ ID NO: 11
DVPADLLKLSLKRRSDRQQFLGH;
SEQ ID NO: 12
SLERNWKLIRDEGLAVMDKAKGL;
SEQ ID NO: 13
GFAKVHYGFILKAQNKIAESIP;
SEQ ID NO: 14
HTGPTNCRLRMHLGLVIPKEGC;
SEQ ID NO: 15
DSFEHEVWQDASSFRLIFIVDVW;
SEQ ID NO: 16
MRGSLLTLQRLVQLFPNDTSLKN;
SEQ ID NO: 17
PQQEDDEFLMATDVDDRFETL;
SEQ ID NO: 18
DGRFYFHLGDAMQRVGNKEAY;
SEQ ID NO: 19
GLSGTSFFTWFMVIALLGVWTSVAGGSGGGFAKVHYGFILKAQNKIAESI
P;
SEQ ID NO: 20
KVYEEVLSVE EVLSVTPNTP NDGFAKVGFA KVHYGFKVHY
GFILKKIAES IPYL;
SEQ ID NO: 21
GTDDGRFYFRVGNKEAYKASVWQRSLYSLYNVNGLK;
SEQ ID NO: 22
SFFTWFMVIALLGVWTSVA;
SEQ ID NO: 23
PADLLKLSLKRRSDRQQF;
SEQ ID NO: 24
GFAKVHYGFILKAQNKIAESIPY;
SEQ ID NO: 25
TGPTNCRLRMHLGLVIPKEGC;
SEQ ID NO: 26
FEHEVWQDASSFRLIFVDVWHPEL;
SEQ ID NO: 27
EHVEGEDLQQEDGPTGEPQQEDDEFL;
SEQ ID NO: 28
PYLKEGIESGDPGTDDGR;
SEQ ID NO: 29
GLKAQPWWTPKETGYTE;
SEQ ID NO: 30
LKAQNKIAESIPYLKEGI;
SEQ ID NO: 31
HLGDAMQRVGNKEAYKWYELGHKRGHFASVW;
SEQ ID NO: 32
VDVWHPELTPQQRRSLPAI;
SEQ ID NO: 33
KNAKSSGNSSSSGSGSGSTSAGSSSPGARRE;
SEQ ID NO: 34
IYDADGDGDFDVDDAKVLLGLKERSTSEPAVP;
SEQ ID NO: 35
EEMM SEQENPDSSEPVVE;
or
SEQ ID NO: 36
FPVEEQQEVPPETNRKTDDPEQKAKVKKKK.
(ii) a polypeptide having an amino acid sequence consisting of a fragment of a polypeptide selected from SEQ ID NO: 1-SEQ ID NO: 36, wherein the fragment is no more than 1, 2, 3 or 4 amino acids shorter than the polypeptide selected from SEQ ID NO: 1-SEQ ID NO: 36;
(iii) a chimeric polyepitope comprising 2, 3 or 4 polypeptides having an amino acid sequence consisting of, or consisting essentially of a polypeptide selected from SEQ ID NO: 1-SEQ ID NO: 36, wherein the polypeptides are covalently connected through one or more linkers; and
(iv) a polypeptide having an amino acid sequence consisting of, or consisting essentially of SEQ ID NO: 38—AABH Fragment 1 or SEQ ID NO: 39—AABH Fragment 2;
b) isolating B cells from the animal;
c) fusing the isolated B cells with HAT-sensitive myeloma cells to produce hybridoma cells; and
d) selecting HAT (hypoxanthine-aminopterin-thymidine) tolerant hybridoma cells that produce antibodies that bind to the polypeptide.

2. The method of claim 1, wherein immunizing comprises injecting the animal with the polypeptide a plurality of times over a plurality of weeks.

3. The method of claim 1, wherein the B cells are isolated from the animal's spleen.

4. The method of claim 1, wherein immunizing comprises in vivo electroporation of the polypeptide.

5. The method of claim 1, wherein fusing comprises use of an electric field or exposure to polyethylene glycol.

6. The method of claim 1, further comprising:
e) cloning the selected hybridoma cells.

7. The method of claim 1, further comprising:
e) isolating monoclonal antibodies from the selected hybridoma cells.

8. A polypeptide having an amino acid sequence consisting of, or consisting essentially of:
SEQ ID NO: 1
CRRGQIKYS;
SEQ ID NO: 2
GPTNCRLRMHLGLVI;
SEQ ID NO: 3
RT WEEGKVLIFD;
SEQ ID NO: 4
WQD ASSFRLIFI;
SEQ ID NO: 5
LS GTSFFTWFMVIALLGVWTSV;
SEQ ID NO: 6
VYEEVLSVTPNDGFAKVHYGFILKAQNK;
SEQ ID NO: 7
FASVWQRSLYNVNGLK;
SEQ ID NO: 8
GLSGTSFFTWFMVIALLGVWTSVA;
SEQ ID NO: 9
GDGDFDVDDAKVLLGLKERSTSEPAV;
SEQ ID NO: 10
IEEAVNAFKELVRKYPQSPRARYGKAQC;
SEQ ID NO: 11
DVPADLLKLSLKRRSDRQQFLGH;
SEQ ID NO: 12
SLERNWKLIRDEGLAVMDKAKGL;
SEQ ID NO: 13
GFAKVHYGFILKAQNKIAESIP;
SEQ ID NO: 14
HTGPTNCRLRMHLGLVIPKEGC;
SEQ ID NO: 15
DSFEHEVWQDASSFRLIFIVDVW;
SEQ ID NO: 16
MRGSLLTLQRLVQLFPNDTSLKN;
SEQ ID NO: 17
PQQEDDEFLMATDVDDRFETL;
SEQ ID NO: 18
DGRFYFHLGDAMQRVGNKEAY;
SEQ ID NO: 19
GLSGTSFFTWFMVIALLGVWTSVAGGSGGGFAKVHYGFILKAQNKIAESI
P;
SEQ ID NO: 20
KVYEEVLSVE EVLSVTPNTP NDGFAKVGFA KVHYGFKVHY
GFILKKIAES IPYL;
SEQ ID NO: 21
GTDDGRFYFRVGNKEAYKASVWQRSLYSLYNVNGLK;
SEQ ID NO: 22
SFFTWFMVIALLGVWTSVA;
SEQ ID NO: 23
PADLLKLSLKRRSDRQQF;
SEQ ID NO: 24
GFAKVHYGFILKAQNKIAESIPY;
SEQ ID NO: 25
TGPTNCRLRMHLGLVIPKEGC;
SEQ ID NO: 26
FEHEVWQDASSFRLIFVDVWHPEL;
SEQ ID NO: 27
EHVEGEDLQQEDGPTGEPQQEDDEFL;
SEQ ID NO: 28
PYLKEGIESGDPGTDDGR;
SEQ ID NO: 29
GLKAQPWWTPKETGYTE;
SEQ ID NO: 30
LKAQNKIAESIPYLKEGI;
SEQ ID NO: 31
HLGDAMQRVGNKEAYKWYELGHKRGHFASVW;
SEQ ID NO: 32
VDVWHPELTPQQRRSLPAI;
SEQ ID NO: 33
KNAKSSGNSSSSGSGSGSTSAGSSSPGARRE;
SEQ ID NO: 34
IYDADGDGDFDVDDAKVLLGLKERSTSEPAVP;
SEQ ID NO: 35
EEMM SEQENPDSSEPVVE;
or
SEQ ID NO: 36
FPVEEQQEVPPETNRKTDDPEQKAKVKKKK.

9. The polypeptide of claim 8, in substantially pure form.

10. A polypeptide having an amino acid sequence consisting of a fragment of a polypeptide selected from SEQ ID NO: 1-SEQ ID NO: 36, wherein the fragment is no more than 1, 2, 3 or 4 amino acids shorter than the polypeptide selected from SEQ ID NO: 1-SEQ ID NO: 36.

11. A polypeptide having an amino acid sequence consisting of, or consisting essentially of:
SEQ ID NO: 38—AABH Fragment 1; or
SEQ ID NO: 39—AABH Fragment 2.

12. A chimeric polyepitope comprising:
2, 3 or 4 polypeptides having an amino acid sequence consisting of, or consisting essentially of:
SEQ ID NO: 1
CRRGQIKYS;
SEQ ID NO: 2
GPTNCRLRMHLGLVI;
SEQ ID NO: 3
RT WEEGKVLIFD;
SEQ ID NO: 4
WQD ASSFRLIFI;
SEQ ID NO: 5
LS GTSFFTWFMVIALLGVWTSV;
SEQ ID NO: 6
VYEEVLSVTPNDGFAKVHYGFILKAQNK;
SEQ ID NO: 7
FASVWQRSLYNVNGLK;
SEQ ID NO: 8
GLSGTSFFTWFMVIALLGVWTSVA;
SEQ ID NO: 9
GDGDFDVDDAKVLLGLKERSTSEPAV;
SEQ ID NO: 10
IEEAVNAFKELVRKYPQSPRARYGKAQC;
SEQ ID NO: 11
DVPADLLKLSLKRRSDRQQFLGH;
SEQ ID NO: 12
SLERNWKLIRDEGLAVMDKAKGL;
SEQ ID NO: 13
GFAKVHYGFILKAQNKIAESIP;
SEQ ID NO: 14
HTGPTNCRLRMHLGLVIPKEGC;
SEQ ID NO: 15
DSFEHEVWQDASSFRLIFIVDVW;
SEQ ID NO: 16
MRGSLLTLQRLVQLFPNDTSLKN;
SEQ ID NO: 17
PQQEDDEFLMATDVDDRFETL;
SEQ ID NO: 18
DGRFYFHLGDAMQRVGNKEAY;
SEQ ID NO: 19
GLSGTSFFTW FMVIALLGVW TSVAGGSGGG FAKVHYGFIL
KAQNKIAESI P;
SEQ ID NO: 20
KVYEEVLSVE EVLSVTPNTP NDGFAKVGFA KVHYGFKVHY
GFILKKIAES IPYL;
SEQ ID NO: 21
GTDDGRFYFRVGNKEAYKASVWQRSLYSLYNVNGLK;
SEQ ID NO: 22
SFFTWFMVIALLGVWTSVA;
SEQ ID NO: 23
PADLLKLSLKRRSDRQQF;
SEQ ID NO: 24
GFAKVHYGFILKAQNKIAESIPY;
SEQ ID NO: 25
TGPTNCRLRMHLGLVIPKEGC;
SEQ ID NO: 26
FEHEVWQDASSFRLIFVDVWHPEL;
SEQ ID NO: 27
EHVEGEDLQQEDGPTGEPQQEDDEFL;
SEQ ID NO: 28
PYLKEGIESGDPGTDDGR;
SEQ ID NO: 29
GLKAQPWWTPKETGYTE;
SEQ ID NO: 30
LKAQNKIAESIPYLKEGI;
SEQ ID NO: 31
HLGDAMQRVGNKEAYKWYELGHKRGHFASVW;
SEQ ID NO: 32
VDVWHPELTPQQRRSLPAI;
SEQ ID NO: 33
KNAKSSGNSSSSGSGSGSTSAGSSSPGARRE;
SEQ ID NO: 34
IYDADGDGDFDVDDAKVLLGLKERSTSEPAVP;
SEQ ID NO: 35
EEMM SEQENPDSSEPVVE;
or
SEQ ID NO: 36
FPVEEQQEVPPETNRKTDDPEQKAKVKKKK.
wherein the polypeptides are covalently connected through one or more linkers.

13. The chimeric polyepitope of claim 11, consisting of, or consisting essentially of an amino acid sequence selected from:
(SEQ ID NO: 40)
GLSGTSFFTWFMVIALLGVWTSVAGGSGGGFAKVHYGFILKAQNKIAESI
PGGSGGHTGPTNCRLRMHLGLVIPKEGC;
(SEQ ID NO: 41)
GLSGTSFFTWFMVIALLGVWTSVAGGSGGIEEAVNAFKELVRKYPQSPRA
RYGKAQCGGSGGDVPADLLKLSLKRRSDRQQFLGH;
(SEQ ID NO: 42)
DVPADLLKLSLKRRSDRQQFLGHGGSGGSLERNWKLIRDEGLAVMDKAKG
L;
and
(SEQ ID NO: 43)
GLSGTSFFTWFMVIALLGVWTSVAGGSGGGFAKVHYGFILKAQNKIAESI
P.

14. A monoclonal antibody that specifically binds to a polypeptide of claim 8, or claim 10, or a chimeric polyepitope of claim 11.

15. The monoclonal antibody of claim 14, that binds to aspartyl (asparaginyl) β-hydroxylase (“AABH”).

16. The monoclonal antibody of claim 14, which is a humanized monoclonal antibody.

17. The monoclonal antibody of claim 14, which is monospecific.

18. The monoclonal antibody of claim 14, which is multispecific or bispecific.

19. The monoclonal antibody of claim 14, which is a whole immunoglobulin.

20. The monoclonal antibody of claim 14, which is an antibody fragment, e.g., selected from the group consisting of: Fab, F(ab′)2, Fab′ or single chain Fv.

21. A conjugated monoclonal antibody comprising a monoclonal antibody of claim 14, conjugated with a chemical moiety.

22. The conjugated antibody of claim 21, wherein the chemical moiety is a detectable label.

23. The conjugated antibody of claim 21, wherein the chemical moiety is chemotherapeutic or cytotoxic agent.

24. The conjugated antibody of claim 23, wherein the chemotherapeutic or cytotoxic agent is selected from the group consisting of: alkylating agent, anti-metabolite, antibiotic, hydroxyurea, platinum-based chemotherapeutic agent, taxane, bortezomib, lenalidomine, thalidomide and metanzinoid.

25. A composition comprising an adjuvant and a monoclonal antibody or conjugated monoclonal antibody according to any one of claims 14-24.

26. The composition of claim 25, wherein the composition is pharmaceutically acceptable.

27. An article comprising a monoclonal antibody or conjugated monoclonal antibody according to any one of claims 14-24 affixed to a solid support

28. The article of claim 27, wherein the solid support is selected from the group consisting of a microtiter plate, an assay plate, an assay well, a nitrocellulose membrane, a bead, a dipstick, and a component of an elution column.

29. A kit comprising:
a) a container comprising a first monoclonal antibody or conjugated monoclonal antibody according to any one of claims 14-24, and
b) one or more of:
(i) a container comprising a second, different monoclonal antibody or conjugated monoclonal antibody according to any one of claims 14-24,
(ii) an assay plate with or without antibody attached, and
(iii) reagents for performing an immunoassay.

30. The kit of claim 29, comprising:
(i) a container comprising a second, different monoclonal antibody or conjugated monoclonal antibody according to any one of claims 14-24.

31. The kit of claim 30, wherein the second monoclonal antibody or conjugated monoclonal antibody comprises a detectable label.

32. The kit of claim 29, comprising:
(ii) an assay plate with or without antibody attached.

33. The kit of claim 29, comprising:
(iii) reagents for performing an immunoassay.

34. A complex comprising a monoclonal antibody of any one of claims 14-20, bound to a polypeptide selected from:
(i) a polypeptide having an amino acid sequence consisting of, or consisting essentially of a polypeptide selected from SEQ ID NO: 1 to SEQ ID NO: 36;
(ii) a polypeptide having an amino acid sequence consisting of a fragment of a polypeptide selected from SEQ ID NO: 1 to SEQ ID NO: 36, wherein the fragment is no more than 1, 2, 3 or 4 amino acids shorter than the polypeptide selected from SEQ ID NO: 1 to SEQ ID NO: 36;
(iii) a chimeric polyepitope comprising 2, 3 or 4 polypeptides having an amino acid sequence consisting of, or consisting essentially of a polypeptide selected from SEQ ID NO: 1-SEQ ID NO: 36, wherein the polypeptides are covalently connected through one or more linkers; and
(iv) a polypeptide having an amino acid sequence consisting of, or consisting essentially of SEQ ID NO: 38—AABH Fragment 1 or SEQ ID NO: 39—AABH Fragment 2; and
(v) AABH.

35. The complex of claim 34, wherein the polypeptide comprises a plurality of amino acid sequences selected from the group consisting of SEQ ID NOs. 1-36.

36. The complex of claim 34, wherein the polypeptide is aspartyl (asparaginyl) β-hydroxylase (“AABH”).

37. A method of making a hybridoma that produces antibodies against aspartyl (asparaginyl) β-hydroxylase (“AABH”) comprising:
a) immunizing an animal with a polypeptide of claim 1 or polyepitope of claim 2 to elicit an immune response;
b) isolating B cells from the animal;
c) fusing the isolated B cells with HAT-sensitive myeloma cells to produce hybridoma cells; and
d) selecting HAT (hypoxanthine-aminopterin-thymidine) tolerant hybridoma cells that produce antibodies that bind to the polypeptide.

38. The method of claim 37, wherein immunizing comprises injecting the animal with the polypeptide a plurality of times over a plurality of weeks.

39. The method of claim 37, wherein the B cells are isolated from the animal's spleen.

40. The method of claim 37, wherein immunizing comprises in vivo electroporation of the polypeptide.

41. The method of claim 37, wherein fusing comprises use of an electric field or exposure to polyethylene glycol.

42. The method of claim 37, further comprising:
e) cloning the selected hybridoma cells.

43. The method of claim 37, further comprising:
e) isolating monoclonal antibodies from the selected hybridoma cells.

44. A method of eliciting antibodies in a mammalian subject comprising administering to the subject a polypeptide of claim 1 or a chimeric polyepitope of claim 11.

45. The method of claim 44, wherein the mammalian subject is selected from mouse, rat, goat, sheep, primate, or human.

46. A hybridoma that produces a monoclonal antibody of any one of claims 14-20.

47. A nucleic acid molecule comprising an expression construct comprising a nucleotide sequence encoding an immunoglobulin polypeptide of a monoclonal antibody of any one of claims 14-20.

48. The nucleic acid molecule of claim 47, comprising an expression construct comprising an expression control sequence operably linked to the nucleotide sequence.

49. The nucleic acid molecule of claim 48, that is comprised in an expression vector.

50. A host cell comprising one or more expression constructs, each expression construct comprising and expression control sequence operatively linked with a nucleotide sequence encoding an immunoglobulin chain of a monoclonal antibody of any one of claims 14-20.

51. The host cell of claim 50, wherein the host cell is selected from the group consisting of a fungal cell, an insect cell and a mammalian cell.

52. The host cell of claim 51, that is a Chinese Hamster Ovary (CHO) cell.

53. A process for creating a monoclonal antibody comprising culturing a hybridoma producing a monoclonal antibody of any one of claims 14-24 or a host cell of claim 50.

54. A method comprising detecting aspartyl (asparaginyl) β-hydroxylase (“AABH”) with an immunoassay that uses a monoclonal antibody of any one of claims 14-24.

55. The method of claim 54, wherein the immunoassay is an ELISA assay, a radioimmunoassay or a sandwich immunoassay.

56. A method of detecting aspartyl (asparaginyl) β-hydroxylase (“AABH”) in a sample comprising contacting the sample with a monoclonal antibody of any one of claims 14-20, and detecting binding of the antibody to AABH.

57. A method of diagnosing cancer in a subject comprising contacting a biological sample from the subject with a monoclonal antibody of any one of claims 14-24 and determining a quantitative measure of an amount of aspartyl (asparaginyl) β-hydroxylase (“AABH”) in the sample, wherein an amount of AABH above a cutoff value, e.g., 3.6 ng/ml serum, indicates cancer.

58. The method of claim 57, wherein the biological sample is selected from blood, serum, plasma, cerebrospinal fluid (CSF), solid tissue, or biopsy.

59. The method of claim 57, wherein the cancer is selected from lung, liver, colon, pancreas, prostate, ovary, bile duct, and breast.

60. The method of claim 57, wherein the cancer is not detectable in the subject by imaging methods, e.g., selected from PET scan, MRI, X-ray, CAT-Scan and ultrasound.

61. The method of claim 57, wherein the method is capable of detecting one cancer cell in a background of 50,000 normal cells.

62. The method of claim 57, further comprising: administering a therapeutic intervention to the subject diagnosed with cancer.

63. A method of diagnosing cancer in a subject, the method comprising:
(a) providing a biological sample from the subject;
(b) contacting the biological sample with a monoclonal antibody of any one of claims 14-24;
(c) measuring an amount of AABH in the sample based on binding of the antibody to AABH;
(d) determining of the measure is greater than a cut off value, wherein a measure greater than the cut off value provides a diagnosis of cancer, and wherein the cut off value is based on measurements of AABH in a plurality of cancer positive samples and a plurality of cancer negative samples.

64. The method of claim 62, wherein the plurality of positive samples and the plurality of negative samples is each at least 10, at least 20, at least 50 or at least 100.

65. A method for treating cancer in a subject in need thereof, the method comprising administering to the subject, a monoclonal antibody or conjugated monoclonal antibody according to any one of claims 14-24, or a composition according to either of claim 25 or 26.

66. The method of claim 65, wherein treatment comprises inhibiting metastasis.

67. A method for immunizing a subject against AABH comprising administering to the subject, one or more of the polypeptides or chimeric polyepitopes of claim 1 or claim 3.

68. The method of any of claim 65 or 67, wherein the administration is parenteral.

69. The method of any of claim 65 or 67, wherein the administration is intravenous.

70. A nucleic acid comprising a nucleotide sequence encoding a polypeptide or chimeric polyepitope of claim 1 or claim 3.

71. An expression vector comprising an expression control sequence operatively linked with a nucleic acid of claim 70.

72. A host cell comprising the expression vector of claim 71.

73. The host cell of claim 72, wherein the cell is a mammalian cell, e.g., a CHO cell.

74. A vaccine comprising one or a plurality of the polypeptides or chimeric polyepitopes of claim 1 or claim 3.

75. A method for determining the presence of a malignant cell in a tissue section, the method comprising:
(a) contacting the section with a monoclonal antibody or conjugated monoclonal antibody according to any one of claims 14-24; and
(b) testing for binding of the antibody to the surface of cells in the tissue section;
wherein a cell whose surface is bound by the antibody is malignant.

76. A method for determining the presence of a malignant cell in a subject, the method comprising:
(a) administering to the subject a monoclonal antibody according to any one of claims 14-24 conjugated with a detectable moiety; and
(b) detecting binding of the antibody to a cell in the subject by an imaging method;
wherein detection of binding a cell indicates that the cell is malignant.

77. A method comprising:
(a) administering to a subject determined to have a level of AABH above a diagnostic cut off for cancer, a therapeutic intervention for the cancer.

78. The method of claim 77, wherein the level is determined from a blood sample from the subject.

79. The method of claim 77, wherein the diagnostic cut off is at least 0.25 ng/ml, 0.3 ng/ml or 0.3 ng/ml.

80. The method of claim 77, wherein the diagnostic cut off has a specificity of at least 90%, at least 95%, at least 99% or at least 99.5%.

81. The method of claim 77, wherein the diagnostic cut off has a sensitivity of at least 90%, at least 95%, at least 99% or at least 99.5%.

82. The method of claim 77, wherein the diagnostic cut off has a precision of at least 90%, at least 95%, at least 99% or at least 99.5%.

83. The method of claim 77, wherein the therapeutic intervention comprises administration of chemotherapy, radiation therapy or immunotherapy.
